(19) United States
(12) Patent Application Publication
(10) Pub. No.: US 2010/0222335 A1
(43) Pub. Date: Sep. 2, 2010

(54) Process For The Preparation Of N,N'-Disubstituted Oxabispidines

(75) Inventors: David Cladingboel, Leicestershire (GB); Gareth Ensor, Leicestershire (GB)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/993,027

(22) PCT Filed: Jun. 12, 2006
(86) PCT No.: PCT/SE2006/000690

§ 371 Date: Dec. 19, 2007

(30) Foreign Application Priority Data
Jun. 20, 2005 (SE) 0501427-9
Dec. 15, 2005 (SE) 0502771-9

(51) Int. Cl. A61K 31/5375; C07D 498/08; A61P 9/06

(52) U.S. Cl. 514/230.5; 544/74

(57) ABSTRACT

There is provided a process for the preparation of a sulfonic acid salt of formula I, or a solvate thereof, which process comprises hydrogenating a sulfonic acid salt of formula II, or a solvate thereof; in the presence of a solvent system consisting essentially of water, a C₃₋₅ secondary alkyl alcohol and no more than 15% v/v of another organic solvent, wherein the sulfonic acid salt of formula I is optionally, without isolation, converted to a compound of formula IX, or a pharmaceutically-acceptable derivative thereof, wherein R¹, R², R³, R⁶, R⁷, R⁸, A, B and D have meanings given in the description.

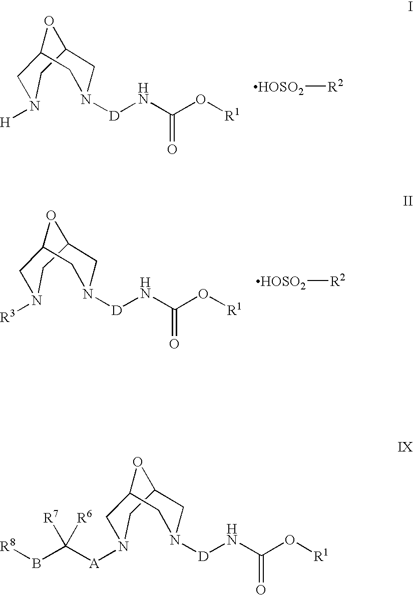

### FIELD OF THE INVENTION

The invention relates to a novel process for the preparation of N,N′-disubstituted oxabispidines in which one of the N-substituents is an (alkoxycarbonylamino)-alkyl group.

### BACKGROUND AND PRIOR ART

In the preparation of drug substances, it is desirable to minimise the cost of producing the substance whilst, at the same time, utilising a preparative route that meets modern environmental and health and safety standards.

Modifications to a preparative route that could result in a decreased overall cost include:
(a) improvements in the yield(s) of one or more steps;
(b) a reduction in the number of synthetic steps and/or unit operations used;
(c) a decrease in the quantities of reagents and/or solvents employed; and/or
(d) minimisation of the amount of energy expended (e.g. through elimination or reduction of the need for heating or cooling); and/or
(e) a shortening of the total time required to complete the preparative route.

Oxabispidine compounds that are useful in the treatment of cardiac arrhythmias are described in WO 01/028992. Amongst the compounds disclosed in that document are certain N,N′-disubstituted oxabispidines in which one of the N-substituents is a 2-(alkoxycarbonylamino)alkyl group. Preparative routes to these compounds are described in WO 01/028992, WO 02/083690, WO 02/028864 and WO 2004/035592.

In the above-mentioned documents, one route to the target N,N′-disubstituted oxabispidines involves the preparation of a mono-substituted oxabispidine. In certain embodiments of this route (e.g. as described in WO 02/083690, WO 02/028864 and WO 2004/035592) the mono-substituted oxabispidine:
(i) has an N-substituent that is a 2-(alkoxycarbonylamino)alkyl group; and
(ii) is obtained by deprotection of an oxabispidine that bears a protective group (e.g. a benzyl group) at the other ring N-atom.

In these embodiments, the final step in the preparation of the target N,N′-disubstituted oxabispidines (coupling of the mono-substituted oxabispidine with a second N-substituent) is performed under a number of different conditions. The exact nature of the conditions employed depend, inter alia, on the precise nature of the reactant providing the second N-substituent, as well as the form in which the mono-substituted oxabispidine is utilised.

For example, WO 02/083690 describes the coupling of neutral [2-(9-oxa-3,7-diazabicyclo[3.3.1]non-3-yl)ethyl]carbamic acid tert-butyl ester to various side-chains in solvent systems comprising a C₂ or C₃ alcohol (i.e. ethanol or isopropanol). WO 2004/035592, however, describes a procedure that effects the same transformation, but instead utilises water as the solvent and the 2,4,6-trimethylbenzene sulfonic acid salt of the mono-substituted oxabispidine as the starting material.

Certain procedures described in WO 02/083690 (i.e. those involving the preparation of alcoholic solutions of neutral [2-(9-oxa-3,7-diazabicyclo[3.3.1]non-3-yl)ethyl]carbamic acid tert-butyl ester) utilise less solvents than the corresponding procedures of WO 2004/035592. On the other hand, the processes described in WO 2004/035592 (which involve the preparation and isolation of sulfonic acid salts of the mono-substituted oxabispidine) utilise fewer reagents than the corresponding procedures of WO 02/083690.

Thus, from the point of view of minimising overall costs, all of the procedures described in the above-mentioned prior art have certain relative disadvantages.

The applicants have now surprisingly found a new method for preparing the key intermediate of the above-mentioned processes (i.e. the mono-substituted oxabispidine), which method involves hydrogenation, in the presence of water and a C₃₋₅ secondary alkyl alcohol, of a sulfonic acid salt of an N′-protected, N-(alkoxycarbonylamino)alkyl-substituted oxabispidine.

This method is neither disclosed nor suggested by any of the above-mentioned prior art documents and provides, inter alia, for processes that utilise fewer reagents than the processes of WO 02/083690 and fewer solvents than the processes of WO 2004/035592. Furthermore, this new method is capable of providing mono-substituted oxabispidines in a form that is more convenient for subsequent manipulation to N,N′-disubstituted oxabispidines.

### DISCLOSURE OF THE INVENTION

According to a first aspect of the invention, there is provided a process for the preparation of a sulfonic acid salt of formula I,

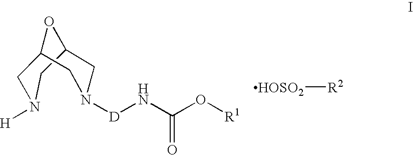

or a solvate thereof;
wherein R¹ represents C₁₋₆ alkyl (optionally substituted by one or more substituents selected from —OH, halo, cyano, nitro and aryl) or aryl;
D represents optionally branched C₂₋₆ alkylene, provided that it does not represent 1,1-C₂₋₆ alkylene;
R² represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy; and
wherein each aryl group, unless otherwise specified, is optionally substituted;
which process comprises hydrogenating a sulfonic acid salt of formula II,

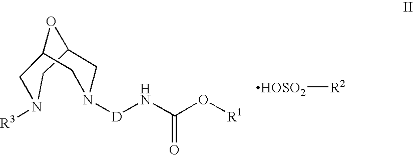

or a solvate thereof;
wherein R³ represents an amino protective group that is labile to hydrogenation, and R¹, R² and D are as defined above,
in the presence of a solvent system consisting essentially of water, a C₃₋₅ secondary alkyl alcohol and no more than 15% v/v of another organic solvent,
which process is hereinafter referred to as “the process of the invention”.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

Unless otherwise specified, alkylene groups as defined herein may be straight-chain or, when there is a sufficient number (i.e. a minimum of two) of carbon atoms, be branched-chain. Such alkylene chains may also be saturated or, when there is a sufficient number (i.e. a minimum of two) of carbon atoms, be unsaturated and/or interrupted by one or more oxygen and/or sulfur atoms. However, such alkylene groups are preferably saturated and not interrupted by any such heteroatoms. Alkylene groups may also be substituted by one or more halo atoms, but are nevertheless preferably not so substituted.

The term “aryl”, when used herein, includes C₆₋₁₃ aryl (e.g. C₆₋₁₀) groups. Such groups may be monocyclic, bicyclic or tricylic and, when polycyclic, be either wholly or partly aromatic. In this respect, C₆₋₁₃ aryl groups that may be mentioned include phenyl, naphthyl, 1,2,3,4-tetrahydronaphthyl, indanyl, indenyl, fluorenyl and the like. For the avoidance of doubt, the point of attachment of substituents on aryl groups may be via any carbon atom of the ring system.

Unless otherwise specified, aryl and aryloxy groups may be substituted by one or more substituents selected from —OH, cyano, halo, nitro, C₁₋₆ alkyl, C₁₋₆ alkoxy, —N(R^{4a})R^{4b}, —C(O)R^{4e}, —C(O)OR^{4d}, —C(O)N(R^{4e})R^{4f}, —N(R^{4g})C(O)R^{4h}, —N(R⁴ⁱ)S(O)₂R^{5a}, —S(O)₂N(R^{4j})(R^{4k}), —S(O)₂R^{5b} and/or —OS(O)₂R^{5c}, (wherein R^{4a} to R^{4k} independently represent H or C₁₋₆ alkyl, or R^{4a} and R^{4b} together represent C₃₋₆ alkylene (resulting in a four- to seven-membered, nitrogen-containing ring) and R^{5a} to R^{5c} independently represent C₁₋₆ alkyl). When substituted, aryl groups are preferably substituted by between one and three substituents.

The term “halo”, when used herein, includes fluoro, chloro, bromo and iodo.

Compounds employed in or produced by the processes described herein (i.e. those involving the process of the invention) may exhibit tautomerism. The process of the invention therefore encompasses the use or production of such compounds in any of their tautomeric forms, or in mixtures of any such forms.

Similarly, the compounds employed in or produced by the processes described herein (i.e. those involving the process of the invention) may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Solvates of the compound of formulae I and II that may be mentioned include hydrates (e.g. monohydrates).

Abbreviations are listed at the end of this specification.

Amino protective groups that are labile to hydrogenation are known to those skilled in the art, and include groups that are described in “Protective Groups in Organic Synthesis”, 3^{rd} edition, T. W. Greene & P. G. M. Wutz, Wiley-Interscience (1999), in particular those mentioned in the chapter entitled “Protection for the Amino Group”, the disclosure in which document is hereby incorporated by reference. Such groups include the Cbz (benzyloxycarbonyl) group and —C(R^{3a})(R^{3b})-aryl groups (in which R^{3a} and R^{3b} independently represent C₁₋₆ alkyl (which alkyl group is optionally substituted by one or more —OH, halo, cyano, nitro and/or aryl) or, preferably, H), such as (benzyl)benzyl groups (e.g. (4-benzyl)benzyl) or, particularly, benzyl groups that are optionally substituted by one or more (e.g. one to three) of the substituents mentioned above in respect of substituents on aryl groups.

Preferred values of R¹ include C₁₋₆ alkyl, particularly saturated C₁₋₆ alkyl.

More preferred values of R¹ include C₃₋₅ alkyl, particularly saturated C₄ alkyl.

Particularly preferred values of R¹ include text-butyl.

Preferred values of D include —(CH₂)₂— and —(CH₂)₃—. In one particular embodiment of the invention, D represents —(CH₂)₂—.

Preferred values of R² include phenyl, optionally substituted by one or more (e.g. one to three) substituents (e.g. three substituents) selected from C₁₋₃ alkyl (e.g. methyl), halo and nitro.

More preferred values of R² include 4-chlorophenyl or, particularly, phenyl, methylphenyl (such as 4-methylphenyl) or trimethylphenyl (such as 2,4,6-trimethylphenyl).

The process of the first aspect of the invention is most preferably performed on a salt of formula II in which R³ represents a benzyl group (optionally substituted as defined above, but most preferably unsubstituted).

Thus, it is preferred that the process according to the first aspect of the invention is performed so as to provide a salt of formula Ia,

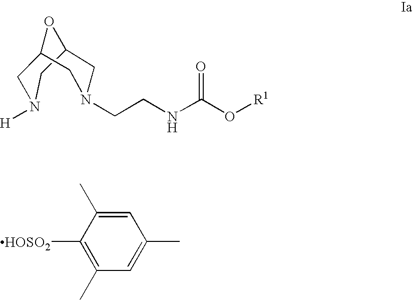

wherein R¹ is as defined above.

In an alternative embodiment, the process according to the first aspect of the invention is performed so as to provide a salt of formula Ib,

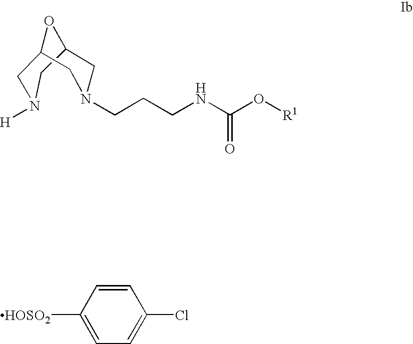

wherein R¹ is as defined above.

The processes described herein (i.e. those involving the process of the invention) should not give rise to stereochemical changes in the reactants or product once formed.

The hydrogenation according to the first aspect of the invention may be performed by methods known to those skilled in the art (e.g. utilising nascent hydrogen), but is preferably effected catalytically (i.e. performed in the presence of a suitable catalyst).

When a catalyst is employed to effect the hydrogenation, it is preferably based on rhodium, ruthenium or, particularly, a platinum group metal (i.e. nickel, platinum or, especially, palladium). The metal upon which the catalyst is based may be employed in powder form, as an oxide or hydroxide or, preferably, dispersed on a suitable support, such as charcoal, activated carbon or other carbon black. Preferably, the catalyst is palladium on charcoal (e.g. 3 to 10% Pd/C, especially 5% Pd/C).

As mentioned above, the process according to the first aspect of the invention is carried out in the presence of a solvent system consisting essentially of water, a C₃₋₅ secondary alkyl alcohol and no more than 15% v/v (e.g. no more than 10, 5, 4, 3, 2 or, particularly 1% v/v) of another organic solvent.

The other organic solvent is preferably not a primary alcohol and is most preferably an acid (e.g. acetic acid) or, particularly, an aprotic solvent (i.e. a solvent lacking an OH group), such as dichloromethane or toluene.

Organic solvents that may be mentioned in this respect include: C₁₋₆ carboxylic acids; di(C₁₋₆ alkyl)ethers (such as di(C₁₋₄ alkyl)ethers, e.g. diethyl ether); C₁₋₆ alkyl acetates (such as C₁₋₄ alkyl acetates, e.g. ethyl acetate); chlorinated hydrocarbons (e.g. chlorinated C₁₋₄ alkanes such as dichloromethane, chloroform and carbon tetrachloride); hexane; petroleum ether: aromatic hydrocarbons, such as benzene and mono-, di- or tri-alkylbenzenes (e.g. mesitylene, xylene, or toluene); and mixtures thereof.

C₃₋₅ secondary alkyl alcohols that may be mentioned include a C₃₋₄ secondary alkyl alcohol such as sec-butanol, iso-butanol or, particularly, isopropanol.

In any event, the volumetric ratio of water to C₃₋₅ secondary alkyl alcohol in the solvent system employed may be any ratio from 5:1 to 1:10, preferably any ratio from 2:1 to 1:7 and, more preferably, any ratio from 1:1 to 1:5, such as 1:3 or thereabouts.

Preferably, the quantity of solvent is between 1 and 4 relative volumes, such as between 1.5 and 2.5 (e.g. about 2) relative volumes.

Hydrogenation may be carried out under a hydrogen atmosphere, either at ambient or elevated pressure (e.g. at least 0.1 MPa (1 bar), such as at least 0.2 MPa (2 bar) and, preferably at least 0.3 MPa (3 bar)). Most preferably, the hydrogenation is carried out at any pressure from 0.2 to 0.4 MPa (e.g. 0.3 to 0.4 MPa, i.e. from 3 to 4 bar), such as at about 0.2 MPa (2 bar) or, particularly, 0.35 MPa (3.5 bar).

Further, the hydrogenation is preferably carried out at a temperature of 5° C. or above (e.g. 10, 15, 20, 25, 30 or, particularly, 35° C. or above), such as any temperature from 15 to 90° C., e.g. from 20, 25, 30 or 35 to 75° C., or, particularly, from 50 to 70° C. (e.g. at about 55 or 65° C.).

After the hydrogenation process according to the first aspect of the invention is complete, the salt of formula I may be isolated by standard techniques (e.g. by crystallisation, evaporation of solvents and/or filtration).

In a particularly preferred embodiment of the first aspect of the invention, the hydrogenation is performed directly on the sulfonic acid salt of formula II (i.e. in the absence of additional (extraneous) acids and/or bases).

Compounds of formula II in accordance with techniques known to those skilled in the art, such as those described in international patent applications WO 01/028992 and WO 02/083690, the disclosures of which are hereby incorporated by reference.

For example, compounds of formula II may be prepared by reaction of a compound of formula III,

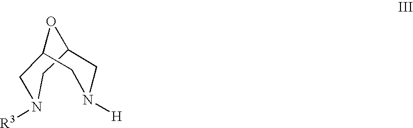

wherein R³ is as defined above, with a compound of formula IV,

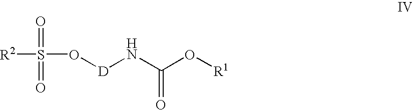

wherein R¹ and R² are as defined above, in an organic solvent (e.g. toluene), for example, under conditions such as those described in WO 02/083690.

Compounds of formulae III and IV may also be prepared in accordance with techniques known to those skilled in the art, such as those described in international patent applications WO 01/028992 and WO 02/083690.

For example, compounds of formula III may be prepared by a dehydrative cyclisation of a compound of formula V,

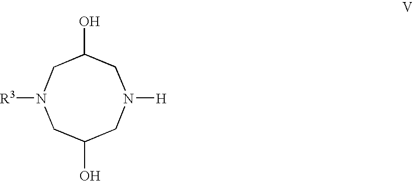

or a protected (e.g. N-benzenesulfonyl or N-nitrobenzenesulfonyl (e.g. N-4-nitrobenzenesulfonyl)) derivative thereof, wherein R¹⁵ is as hereinbefore defined. The cyclisation may be carried out under conditions such as those described in WO 02/083690 (e.g. in the presence of a dehydrating agent, such as a strong acid (e.g. methanesulfonic acid), and a reaction-inert organic solvent (e.g. toluene)).

Compounds of formula III may alternatively be prepared according to, or by analogy with, known techniques, such as reaction of a compound of formula VI,

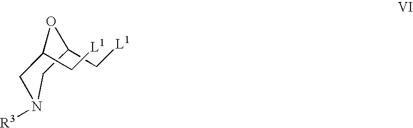

wherein L¹ represents a suitable leaving group (e.g. halo, such as iodo) and R³ is as defined above, with ammonia or a protected derivative thereof (e.g. benzylamine), for example under conditions such as those described in Chem. Ber. 96(11), 2827 (1963).

Compounds of formula IV may be prepared by reaction of a corresponding compound of formula VII,

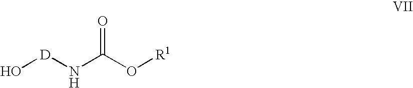

wherein R¹ and D are as hereinbefore defined, with a compound of formula VIII,

R²—S(O)₂-L² VIII

wherein L² represents a leaving group (e.g. halo, such as chloro) and R² is as hereinbefore defined, for example under reaction conditions such as those described in WO 02/083690.

Compounds of formulae V, V, VI, VII and VIII, and derivatives thereof, are either commercially available, are known in the literature (e.g. the preparation of compounds of formulae V and VI is described in WO 02/083690) or may be obtained by conventional synthetic procedures, in accordance with known techniques, from readily available starting materials using appropriate reagents and reaction conditions.

As indicated above, the sulfonic acid salt of formula I may, if desired, be isolated and, optionally, further purified by means of techniques known to those skilled in the art. However, in a particularly preferred embodiment of the invention, the salt of formula I is not isolated, i.e. it is further elaborated without its separation or removal from the solvent system in which it was prepared.

Thus, the process according to the first aspect of the invention is preferably performed so as to provide a solution of a salt of formula I in a solvent system consisting essentially of water, a C₃₋₅ secondary alkyl alcohol and no more than 20% (e.g. no more than 15 or, particularly, 10 or 5%) v/v of another organic solvent.

Preferred solvent systems in this embodiment include those described above, such as a solvent system consisting essentially of water, isopropanol and no more than 15% v/v of another organic solvent.

When the salt of formula I is not isolated (as described above), the solvent system in which it resides may be compatible with processes for coupling the salt of formula Ito a molecule providing a N′-substituent. In these instances, the process by which the resulting N,N′-disubstituted oxabispidine is prepared (from the compound of formula II) is particularly efficient compared to the processes described in the prior art.

In this respect, and according to a second aspect of the present invention, there is provided a process for the preparation of a compound of formula IX,

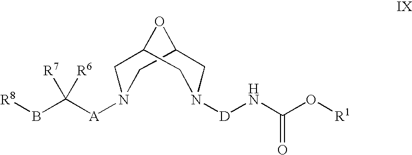

or a pharmaceutically-acceptable derivative thereof;
wherein R¹ and D are as hereinbefore defined;
R⁶ represents H, halo, C₁₋₆ alkyl, —OR⁹, -E-N(R¹⁰)R¹¹ or, together with R⁷, represents ═O;
R⁷ represents H, C₁₋₆ alkyl or, together with R⁶, represents ═O;
R⁹ represents H, C₁₋₆ alkyl, -E-aryl, -E-Het¹, —C(O)R^{12a}, —C(O)OR^{12b} or —C(O)N(R^{13a})R^{13b};
R¹⁰ represents H, C₁₋₆ alkyl, -E-aryl, -E-Het¹, —C(O)R^{12a}, —C(O)OR^{12b}, —S(O)₂R^{12e}, —[C(O)]ₚN(R^{13a})R^{13b} or —C(NH)NH₂;
R¹¹ represents H, C₁₋₆ alkyl, -E-aryl or —C(O)R^{12d};
R^{12a} to R^{12d} independently represent, at each occurrence when used herein, C₁₋₆ alkyl (optionally substituted by one or more substituents selected from halo, aryl and Het²), aryl, Het³, or R^{12a} and R^{12d} a independently represent H;

R^{13a} and R^{13b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;

E represents, at each occurrence when used herein, a direct bond or C₁₋₄ alkylene;
p represents 1 or 2;
A represents a direct bond -J-, -J-N(R^{14a})—, -J-S(O)₂N(R^{14b})—, -J-N(R^{14c})S(O)₂— or -J-O— (in which latter four groups, -J is attached to the oxabispidine ring nitrogen);
B represents —Z—{[C(O)]ₐC(H)(R^{15a})}_{b}—, —Z—[C(O)]_{c}N(R^{15b})—, —Z—N(R^{15c})S(O)₂—, —Z—S(O)₂N(R^{15d})—, —Z—O— (in which latter six groups, Z is attached to the carbon atom bearing R⁶ and R⁷), —N(R^{15e})—Z—, —N(R^{15f})S(O)₂—Z—, —S(O)₂N(R^{15g})—Z— or —N(R^{15h})C(O)O—Z— (in which latter four groups, Z is attached to the R⁸ group);
J represents C₁₋₆ alkylene optionally interrupted by —S(O)₂N(R^{14d})— or —N(R^{14e})S(O)₂— and/or optionally substituted by one or more substituents selected from —OH, halo and amino;
Z represents a direct bond or C₁₋₄ alkylene, optionally interrupted by —N(R¹⁵ⁱ)S(O)₂— or —S(O)₂N(R^{15j})—;
a, b and c independently represent 0 or 1;
n represents 0, 1 or 2;
R^{14a} to R^{14e} independently represent, at each occurrence when used herein, H or C₁₋₆ alkyl;
R^{15a} represents H or, together with a single ortho-substituent on the R⁸ group (ortho-relative to the position at which the B group is attached), R^{15a} represents C₂₋₄ alkylene optionally interrupted or terminated by O, S, N(H) or N(C₁₋₆ alkyl);
R^{15b} represents H, C₁₋₆ alkyl or, together with a single ortho-substituent on the R⁸ group (ortho-relative to the position at which the B group is attached), R^{15b} represents C₂₋₄ alkylene;
R^{15c} to R^{15j} independently represent, at each occurrence when used herein, H or C₁₋₆ alkyl;
R⁸ represents phenyl or pyridyl, both of which groups are optionally substituted by one or more substituents selected from —OH, cyano, halo, nitro, C₁₋₆ alkyl (optionally terminated by —N(H)C(O)OR^{16a}), C₁₋₆ alkoxy, —N(R^{17a})R^{17b}, —C(O)R^{17c}, —C(O)OR^{17d}, —C(O)N(R^{17e})R^{17f}, —N(R^{17g})C(O)R^{17h}, —N(R¹⁷ⁱ)C(O)N(R^{17j})R^{17k}, —N(R^{17m})S(O)₂R^{16b}, —S(O)₂N(R¹⁷ⁿ)R^{16o}, —S(O)₂R^{16c}, —OS(O)₂R^{16d} and/or aryl; and an ortho-substituent (ortho-relative to the attachment of B) may
(i) together with R^{15a}, represent C₂₋₄ alkylene optionally interrupted or terminated by O, S, N(H) or N(C₁₋₆ alkyl), or
(ii) together with R^{15b}, represent C₂₋₄ alkylene;
   R^{16a} to R^{16d} independently represent C₁₋₆ alkyl;
   R^{17a} and R^{17b} independently represent H, C₁₋₆ alkyl or together represent C₃₋₆ alkylene, resulting in a four- to seven-membered nitrogen-containing ring;
   R^{17c} to R^{17o} independently represent H or C₁₋₆ alkyl; and
   Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{18a})R^{18b}, —C(O)R^{18c}, —C(O)OR^{18d}, —C(O)N(R^{18e})R^{18f}, —N(R^{18g})C(O)R^{18h}, —S(O)₂N(R¹⁸ⁱ)(R^{18j}) and/or —N(R^{18k})S(O)₂R^{18l};
   R^{18a} to R^{18l} independently represent C₁₋₆ alkyl, aryl or R^{18a} to R^{18k} independently represent H;
   provided that:
(a) when R⁷ represents H or C₁₋₆ alkyl; and
   A represents -J-N(R^{14a})— or -J-O—, then:
      (i) J does not represent C₁ alkylene or 1,1-C₂₋₆ alkylene; and
      (ii) B does not represent —N(R^{15b})—, —N(R^{15c})S(O)₂—, —S(O)ₙ—, —O—, —N(R^{15e}), —N(R^{15f})S(O)₂—Z— or —N(R^{15h})C(O)O—Z—; and
(b) when R² represents —OR⁹ or -E-N(R¹⁰)R¹¹ in which E represents a direct bond, then:
   (i) A does not represent a direct bond, -J-N(R^{14a})—, -J-S(O)₂—N(R^{14b})— or -J-O—; and
   (ii) B does not represent —N(R^{15b})—, —N(R^{15c}S(O)₂—, —S(O)ₙ—, —O—, —N(R^{15e})—Z, —N(R^{15f})S(O)₂—Z— or —N(R^{15h})C(O)O—Z—; and
(c) when A represents) -J-N(R^{14c})S(O)₂—, then J does not represent C₁ alkylene or 1,1-C₂₋₆ alkylene; and
(d) when R³ represents H or C₁₋₆ alkyl and A represents -J-S(O)₂N(R^{14b})—, then B does not represent —N(R^{15b})—, —N(R^{15c})S(O)₂—, —S(O)ₙ—, —O—, —N(R^{15e})—Z—, —N(R^{15f})S(O)₂—Z— or —N(R^{15h})C(O)O—Z—; and
   wherein each aryl and aryloxy group, unless otherwise specified, is optionally substituted;
   which process comprises:
(I) hydrogenating a sulfonic acid salt of formula II,

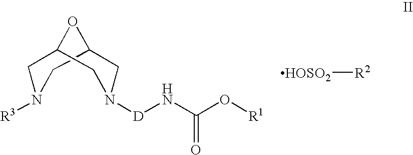

or a solvate thereof;
wherein R¹, R², R³ and D are as defined above,
in the presence of a solvent system consisting essentially of water, a C₃₋₅ secondary alkyl alcohol and no more than 15% v/v of another organic solvent; and
(II) without isolating it, reacting the sulfonic acid salt of formula I thereby formed,

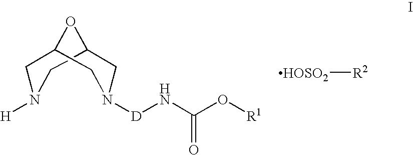

wherein R¹ and D are as hereinbefore defined with base and
(a) a compound of formula X,

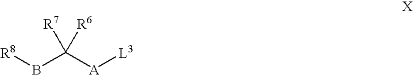

wherein L³ represents a leaving group (e.g. mesylate, tosylate, mesitylenesulfonate or halo) and R⁶, R⁷, R⁸, A and B are as hereinbefore defined, or
(b) for compounds of formula IX in which A represents C₂ alkylene and R² and R³ together represent a ═O group, a compound of formula XI,

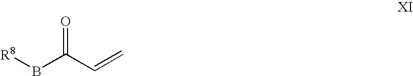

wherein R⁸ and B are as hereinbefore defined, or
(c) for compounds of formula IX in which A represents CH₂ and R⁶ represents —OH or —N(H)R¹⁰, a compound of formula XII,

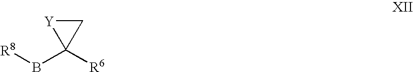

wherein Y represents —O— or —NR¹⁰— and R⁶, R⁸, R¹⁰ and B are as hereinbefore defined,
wherein the reaction with the compound of formula X, XI or XII is carried out in the presence of a solvent system comprising water and a C₃₋₅ secondary alkyl alcohol,
   which process is also hereinafter referred to as “the process of the invention”.

By “without isolating”, we mean that the salt of formula I (which acts as an intermediate) is not separated from the solvent system in which it is formed (i.e. the system consisting essentially of water, a C₃₋₅ secondary alkyl alcohol and no more than 15% v/v of another organic solvent).

In this respect, the term “without isolating” encompasses processes in which at least 10% (e.g. at least 20, 30, 40, 50, 60, 70, 80, 90 or, particularly, 95%) of the solvent employed in step (I) above is carried through and employed in step (II) above. Thus, the mixture of solvents carried over from step (I) above may provide all or, preferably, part of the solvent system employed in step (II) above (i.e. the solvent system comprising water and a C₃₋₅ secondary alkyl alcohol).

The term “aryloxy”, when used herein includes C₆₋₁₃ aryloxy groups such as phenoxy, naphthoxy, fluorenoxy and the like. For the avoidance of doubt, aryloxy groups referred to herein are attached to the rest of the molecule via the O-atom of the oxy-group.

Unless otherwise specified, aryloxy groups may be substituted by one or more substituents selected from —OH, cyano, halo, nitro, C₁₋₆ alkyl, C₁₋₆ alkoxy, —N(R^{4a})R^{4b}, —C(O)R^{4c}, —C(O)OR^{4d}, —C(O)N(R^{4e})R^{4f}, —N(R^{4g})C(O)R^{4h}, —N(R⁴ⁱ)S(O)₂R^{5a}, —S(O)₂N(R^{4j})(R^{4k}), —S(O)₂R^{5b} and/or —OS(O)₂R^{5c}, (wherein R^{4a} to R^{4k} and R^{5a} to R^{5c} are as hereinbefore defined). When substituted, aryloxy groups are preferably substituted by between one and three substituents.

Het (Het¹, Het², Het³, Het⁴ and Het⁵) groups that may be mentioned include those containing 1 to 4 heteroatoms (selected from the group oxygen, nitrogen and/or sulfur) and in which the total number of atoms in the ring system are between five and twelve. Het (Het¹, Het², Het³, Het⁴, and Het⁵) groups may be fully saturated, wholly aromatic, partly aromatic and/or bicyclic in character. Heterocyclic groups that may be mentioned include 1-azabicyclo[2.2.2]octanyl, benzimidazolyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazinonyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo[c]furanyl, 2,3-dihydropyrrolo[2,3-b]pyridyl, dioxanyl, furanyl, hexahydropyrimidinyl, hydantoinyl, imidazolyl, imidazo[1,2-a]pyridyl, imidazo[2,3-b]thiazolyl, indolyl, isoquinolinyl, isoxazolyl, maleimido, morpholinyl, oxadiazolyl, 1,3-oxazinanyl, oxazolyl, phthalazinyl, piperazinyl, piperidinyl, purinyl, pyranyl, pyrazinyl, pyrazolyl, pyridyl, pyrimidinyl, pyrrolidinonyl, pyrrolidinyl, pyrrolinyl, pyrrolo[2,3-b]pyridyl, pyrrolo[5,1-b]pyridyl, pyrrolo[2,3-c]pyridyl, pyrrolyl, quinazolinyl, quinolinyl, sulfolanyl, 3-sulfolenyl, 4,5,6,7-tetrahydrobenz-imidazolyl, 4,5,6,7-tetrahydrobenzopyrazolyl, 5,6,7,8-tetrahydrobenzo[e]pyrimidine, tetrahydrofuranyl, tetrahydropyranyl, 3,4,5,6-tetrahydropyridyl, 1,2,3,4-tetrahydropyrimidinyl, 3,4,5,6-tetrahydro-pyrimidinyl, thiadiazolyl, thiazolidinyl, thiazolyl, thienyl, thieno[5,1-c]pyridyl, thiochromanyl, triazolyl, 1,3,4-triazolo[2,3-b]pyrimidinyl and the like.

Substituents on Het (Het¹, Het², Het³, Het⁴ and Het⁵) groups may, where appropriate, be located on any atom in the ring system including a heteroatom. The point of attachment of Het (Het¹, Het², Het³, Het⁴ and Het⁵) groups may be via any atom in the ring system including (where appropriate) a heteroatom, or an atom on any fused carbocyclic ring that may be present as part of the ring system. Het (Het¹, Het², Het³, Het⁴ and Het⁵) groups may also be in the N- or S-oxidised form.

Pharmaceutically acceptable derivatives of the compound of formula IX include salts and solvates. Salts which may be mentioned include acid addition salts.

Pharmaceutically acceptable derivatives of the compounds of formula IX also include, at the oxabispidine or (when R⁸ represents pyridyl)pyridyl nitrogens, C₁₋₄ alkyl quaternary ammonium salts and N-oxides, provided that when a N-oxide is present:
(a) no Het (Het¹, Het², Het³, Het⁴ and Het⁵) group contains an unoxidised S-atom; and/or
(b) n does not represent 0 when B represents —Z—S(O)ₙ—.

Preferred compounds of formula IX include those in which:

R¹ represents C₁₋₆ alkyl, particularly saturated C₁₋₆ alkyl;
R⁶ represents H, halo, C₁₋₃ alkyl, —OR⁹, —N(H)R¹⁰ or, together with R⁷, represents ═O;
R⁷ represent H, C₁₋₃ alkyl or, together with R⁶, represents ═O;
R⁹ represents H, C₁₋₆ alkyl, -E-(optionally substituted phenyl) or -E-Het¹;
R¹⁰ represents H, C₁₋₆ alkyl, -E-(optionally substituted phenyl), —C(O)R^{12a}, —C(O)OR^{12b}, S(O)₂R^{12c}, —C(O)N(R^{13a})R^{13b} or —C(NH)NH₂;
R^{12a} to R^{12c} independently represent C₁₋₆ alkyl, or R^{12a} represents H;
R^{13a} and R^{13b}) independently represent H or C₁₋₄ alkyl;
E represents, at each occurrence when used herein, a direct bond or C₁₋₂ alkylene;
A represents -J-, -J-N(R^{14a})— or -J-O—;
B represents —Z—, —Z—N(R^{15b})—, —Z—S(O)ₙ— or —Z—O—;
J represents C₁₋₄ alkylene;
Z represents a direct bond or C₁₋₃ alkylene;
R^{14a} and R^{15b} independently represent H or C₁₋₄ alkyl;
n represents 0 or 2;
R⁴ represents phenyl or pyridyl, both of which groups are optionally substituted by one or more substituents selected from cyano, halo, nitro, C₁₋₆ alkyl, C₁₋₆ alkoxy, —NH₂, —C(O)N(R^{17e})R^{17f}, —N(R^{17g})C(O)R^{17h} and —N(R^{17m})S(O)₂—R^{16b};
R^{16b} represents C₁₋₃ alkyl
R^{17e} to R^{17m} independently represent, at each occurrence when used herein, H or C₁₋₄ alkyl;
Het¹ to Het⁵ are optionally substituted by one or more substituents selected from ═O, cyano, halo, nitro, C₁₋₄ alkyl, C₁₋₄ alkoxy, —N(R^{18a})R^{18b}, —C(O)R^{18c} and C(O)OR^{18d};
R^{18a} to R^{18d} independently represent H, C₁₋₄ alkyl or aryl;
R represents —(CH₂)₂—;
optional substituents on aryl and aryloxy groups, are unless otherwise stated, one or more substituents selected from cyano, halo, nitro, C₁₋₄ alkyl and C₁₋₄ alkoxy.

Further preferred compounds of formula IX include those in which:

R¹ represents C₃₋₅ alkyl, particularly saturated C₄ alkyl;
R⁶ represents H, methyl, —OR⁹ or —N(H)R¹⁰;
R⁷ represents H or methyl;
R⁹ represents H, C₁₋₂ alkyl or phenyl (which phenyl group is optionally substituted by one or more substituents selected from cyano and C₁₋₄ alkoxy);
R¹⁰ represents H, C₁₋₂ alkyl, phenyl (which phenyl group is optionally substituted by one or more substituents selected from cyano, halo, nitro, C₁₋₄ alkyl and C₁₋₄ alkoxy), —C(O)—R^{12a} or —C(O)O—R^{12b});
R^{12a} and R^{12b} independently represent C₁₋₆ alkyl;
A represents C₁₋₄ alkylene;
B represents —Z—, —Z—N(R^{15b})—, —Z—S(O)₂— or —Z—O—;
R^{15b} represents H or methyl;
R⁸ represents pyridyl or phenyl, which latter group is optionally substituted by one to three substituents selected from cyano, nitro, C₁₋₂ alkoxy, NH₂ and —N(H)S(O)₂CH₃;

Yet more preferred compounds of formula IX include those in which:

R⁶ represents H, —OR⁹ or —N(H)R¹⁰;
R⁹ represents H or phenyl (optionally substituted by one or more substituents selected from cyano and C₁₋₂ alkoxy);
R¹⁰ represents H, phenyl (optionally substituted by one or more cyano groups) or —C(O)O—C₁₋₅ alkyl;
A represents C₁₋₃ alkylene;
B represents —Z—, —Z—N(H)—, —Z—S(O)₂— or —Z—O—;
R⁸ represents phenyl substituted by cyano in the ortho- and/or, in particular, the para-position relative to B.

Particularly preferred compounds of formula IX include:

R¹ represents tert-butyl;
R⁶ represents H or —OH;
R⁷ represents H;
A represents CH₂;
B represents —Z—, —Z—N(H)— or —Z—O—;
Z represents a direct bond or C₁₋₂ alkylene;
R⁸ represents para-cyanophenyl.

Especially preferred compounds of formula IX include those in which the structural fragment of formula IXa,

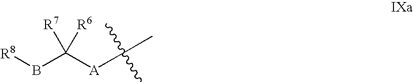

represents:

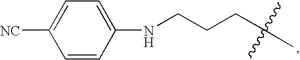

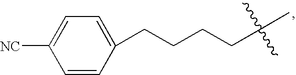

and particularly

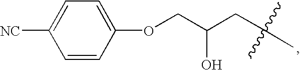

such as

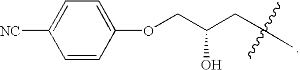

In a further embodiment of the invention, compounds of formula IX that may be mentioned include those in which

R¹ represents tent-butyl;
D represents —(CH₂)₂— or —(CH₂)₃—;
R⁶ represents H or —OH;
R⁷ represents H;
A represents CH₂;
B represents —Z—O—;
Z represents a direct bond or C₁₋₂ alkylene (e.g. CH₂);
R⁸ represents phenyl substituted by cyano in the para-position (relative to B) and optionally substituted by fluoro in the ortho-position (relative to B).

In relation to this further embodiment of the invention, compounds of formula IX that may be mentioned include those in which the structural fragment of formula IXa,

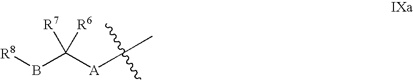

represents:

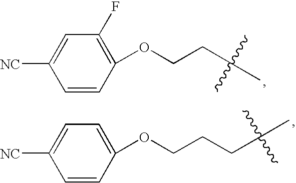

and

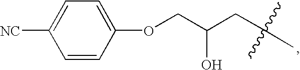

such as

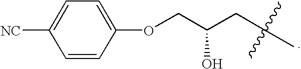

Thus, specific compounds of formula IX that may be mentioned include:
tert-butyl 2-{7-[(2S)-3-(4-cyanophenoxy)-2-hydroxypropyl]-9-oxa-3,7-diaza-bicyclo[3.3.1]non-3-yl}ethylcarbamate;
tert-butyl (2-{7-[2-(4-cyano-2-fluorophenoxy)ethyl]-9-oxa-3,7-diazabicyclo-[3.3.1]non-3-yl}ethyl)carbamate;
tert-butyl (3-{7-[3-(4-cyanophenoxy)propyl]-9-oxa-3,7-diazabicyclo[3.3.1]non-3-yl}propyl)carbamate,
   and salts and/or solvates thereof.

With respect to the process according to the second aspect of the invention, preferred salts of formula II include those defined hereinbefore with respect to the process according to the first aspect of the invention.

Preferences for the temperature, solvent system, and hydrogenation conditions for step (I) above include those defined hereinbefore with respect to the process according to the first aspect of the invention.

As stated above, the solvent system employed in step (II) above comprises water and a C₃₋₅ secondary alkyl alcohol. Preferred solvent systems for step (II) include those that consist essentially of water, a C₃₋₅ secondary alkyl alcohol and no more than 20% v/v (e.g. no more than 15, 10 or, particularly, 5% v/v) of another organic solvent.

Organic solvents that may be mentioned in this respect include the organic solvents mentioned above with respect to the process according to the first aspect of the invention. A particular solvent that may be mentioned is toluene. Those skilled in the art will appreciate that, in the instances where R³ in the salt of formula II is benzyl, toluene is a product of the hydrogenation of step (I) above, (and hence may be present in the solvent system carried over to step (II) above).

Further, when the organic solvent is an acid, those skilled in the art will appreciate that this acid may need to be neutralised (by addition of a base, such as one of those mentioned below with respect to the process according to the second aspect of the invention) before, or at the same time as, the salt of formula I is reacted with the compound of formula X, XI or XII.

When a catalyst is employed in the hydrogenation of step (I), then the mixture obtained after step (I) is substantially complete is preferably filtered to remove the catalyst, before that mixture is employed directly in step (II) above.

It is preferred that step (II) above, i.e. reaction between the salt of formula I and the compound of formula X, XI or XII, is initiated by addition of the salt of formula I (dissolved in the solvent system mentioned above with respect to the process according to the first aspect of the invention) to a mixture of base and the compound of formula X, XI or XII. In this embodiment, the compound of formula X, XI or XII is preferably pre-mixed (e.g. as a solvent-free solid or oil) with base.

In an alternative embodiment, reaction between the salt of formula I and the compound of formula X, XI or XII, is initiated by addition of the compound of formula X, XI or XII to a mixture of base and the salt of formula I (dissolved in the solvent system mentioned above with respect to the process according to the first aspect of the invention).

Base may be employed in the form of a solid or, preferably, in the form of an aqueous solution. The base may be an alkali metal hydrogen carbonate, an alkali metal hydroxide and/or, particularly, an alkali metal carbonate (e.g. potassium carbonate or, particularly, sodium carbonate).

When the base employed is in the form of an aqueous solution, then the molarity is in the range 0.1 to 5 M, preferably between 0.1 and 3 M, such as about 0.3 M.

The quantity of base employed is preferably sufficient to neutralise the salt of formula I (i.e. liberate the corresponding neutral amine) and, if necessary (e.g. for reaction with a compound of formula X), to neutralise any acid that may be generated by the reaction of step (II) above. Thus, where base is required only to neutralise the salt of formula I, the quantity employed should be at least equimolar to the quantity of the salt of formula I employed. Further, where base is required to neutralise the salt of formula I and acid generated during the reaction of step (II) above, then the quantity employed should represent at least two molar equivalents compared to the quantity of the salt of formula I employed.

When a dibasic compound (e.g. potassium carbonate or, particularly, sodium carbonate) is employed as a base, then the stoichiometric ratio of base to the compound of formula I is in the range 2:1 to 1:5, preferably between 1:1 and 1:3, such as 1:2 or thereabouts.

The reaction of step (II) above is preferably between a salt of formula I and a compound of formula XII. In this respect, particularly preferred compounds of formula XII include 4-(oxiranylmethoxy)benzonitrile, such as 4-[(2S)-oxiranyl-methoxy]benzonitrile.

In another embodiment of the invention, the reaction of step (II) is between a salt of formula I and a compound of formula X. Compounds of formula X that may be mentioned in this respect include those in which R⁶ to R⁸, A and B are as defined above and L³ represents mesitylenesulfonate or, particularly, tosylate or halo (e.g. bromo). Specific compounds of formula X that may be mentioned include 4-(2-bromoethoxy)-3-fluorobenzonitrile and 2-(4-cyano-2-fluorophenoxy)ethyl toluene-4-sulfonate.

When a compound of formula XII is employed in the reaction of step (II) above, then the stoichiometric ratio of the compound of formula I to the compound of formula XII is in the range 3:2 to 2:3, such as 1:1 or thereabouts.

Reaction with the compound of formula X, XI or XII may take place at ambient temperature or, preferably, at elevated temperature, such as at any temperature from 30 to 120° C. (e.g. from 60 to 110° C.). When the C₃₋₅ secondary alkyl alcohol employed as part of the solvent system form step (II) above is isopropanol, then the reaction is preferably performed at about 78° C.

When reaction between the salt of formula I and the compound of formula X, XI or XII is substantially complete, then the compound of formula IX may be isolated by way of the following procedure:
(a) removal, by distillation, of substantially all of the alcoholic component of the solvent system;
(b) addition of an organic solvent that is immiscible with water;
(c) subsequent to its addition, washing of that organic solvent with an aqueous solution of base;
(d) extraction of the compound of formula I from the resulting organic phase into an aqueous solution of acid;
(e) basification of the resulting aqueous, acidic phase and extraction of the compound of formula I into an alcoholic solvent that is immiscible with concentrated aqueous sodium chloride solution; and
(f) crystallisation and isolation of the compound of formula I from that alcoholic solvent.

Those skilled in the art will appreciate that, where appropriate in steps (a) to (f) above, separation of immiscible solvent phases may take place.

Distillations that may be undertaken during the work-up (see, for example, step (a) above) may be performed under reduced pressure and/or at elevated temperature (e.g. between 25 and 110° C.).

Examples of non water-miscible organic solvents that may be employed at step (b) above include di(C₁₋₆ alkyl)ethers (such as di(C₁₋₄ alkyl)ethers, e.g. diethyl ether and diisopropyl ether), C₁₋₆ alkyl acetates (such as C₁₋₄ alkyl acetates, e.g. ethyl acetate), chlorinated hydrocarbons (e.g. chlorinated C₁₋₄ alkanes such as dichloromethane, chloroform and carbon tetrachloride), hexane, petroleum ether, and an aromatic hydrocarbon, such as benzene and mono-, di- or tri-alkylbenzenes (e.g. mesitylene, xylene, or toluene). Preferred organic solvents that may be employed include aromatic solvents (e.g. benzene or, particularly, toluene) and di(C₁₋₄ alkyl)ethers (e.g. diisopropyl ether). Such organic solvents may be employed in the work-up at elevated temperature.

The skilled person will appreciate that steps (a) and (b) may be reversed, or that step (b) may be performed both before and after step (a), in the event that the organic solvent employed at (b) above has a boiling point that is higher than that of the solvent system employed in the process of the invention (i.e. the mixture comprising water and a C₃₋₅ secondary alkyl alcohol). For example, when a mixture of water and isopropanol is employed in the process of the invention and toluene is employed at step (b) above, then toluene may be added prior to removal (by way of distillation) of the mixture of water and isopropanol.

Examples of aqueous bases that may be employed in step (c) above include alkali metal hydroxides (e.g. sodium hydroxide). Washing with base (step (c) above) may be performed so as to remove mesitylenesulfonic acid from the product mixture.

It is preferred that the acid employed in step (d) above is a weak and/or a water-soluble acid, particularly both a weak and water-soluble acid.

The term “weak, water-soluble acid”, when used herein, includes references to acids that have a solubility in water of 1 mg/mL or more and a pKa (measured in water) of between 2 and 7 (preferably between 3 and 5). In this respect, preferred water-soluble, weak acids that may be mentioned include carboxylic acids such as acetic or, particularly, citric acid.

The quantity of acid employed in step (d) above is preferably sufficient to extract substantially all of the compound of formula I from the organic phase into the aqueous, acidic phase (e.g. a quantity that is equimolar to the quantity of the compound of formula I). In this manner, the extraction of step (d) may be employed to remove non-basic impurities.

Alcoholic solvents that are immiscible with concentrated aqueous sodium chloride solution (and that may be employed in step (e) above) include 4-methyl-2-pentanol, n-butanol, s-butanol and n-hexanol.

By “concentrated aqueous sodium chloride solution” we include references to solutions of sodium chloride in water that have between 5 and 35 (e.g. 10 or 20) weight percent of NaCl.

The crystallisation of step (f) above may be performed by allowing the solution in alcoholic solvent to stand and/or, if elevated temperature is employed in a previous work-up step, by cooling the solution to, for example, ambient temperature, e.g. any temperature from 10 to 30° C., such as from 17 to 23° C. (e.g. 20° C.). Furthermore, a precipitating solvent (e.g. a dialkyl ether, such as diisopropyl ether) may be added to the alcoholic solution to encourage crystallisation of the compound of formula IX.

In an alternative embodiment, the compound of formula IX may be isolated in acid addition salt form. In this embodiment, the acid addition salt is formed by contacting the compound of formula I with acid, optionally in the presence of a suitable solvent system (e.g. an organic solvent such as isopropyl acetate, ethanol, or a mixture thereof). Particular acid addition salts that may be mentioned include hydrobromic acid and L-tartaric acid salts.

The product that crystallises may be isolated by techniques known to the skilled person, such as filtration, washing with solvent and evaporation of solvent, for example under conditions such as those described hereinafter.

The product may, if desired, be further purified using techniques known to the skilled person, such as those described herein.

As mentioned above, the compounds of formulae X, XI and XII may be pre-mixed with base before they are reacted with a salt of formula I. Such pre-mixing provides the advantage that reaction between the salt of formula I and the compound of formula X, XI or XII may be initiated simply by filtering, directly into the mixture of base and compound of formula X, XI or XII, the solution obtained after the process according to the first aspect of the invention has been performed. This minimises the quantity of solvents and number of vessels required to effect both of the hydrogenation and coupling steps.

As also mentioned above, it is preferred that coupling takes place between the salt of formula I and a compound of formula XII, as hereinbefore defined. Further, preferred bases include aqueous solutions of base.

Thus, according to a third aspect of the invention, there is provided a mixture consisting essentially of:

(1) an aqueous solution of base; and
(2) a compound of formula XII, as hereinbefore defined.

In respect of this aspect of the invention, preferences for the base and the compound of formula XII are as defined above. In particular, it is preferred that the base is an alkali metal carbonate (such as sodium carbonate) and the compound of formula XII is 4-(oxiranylmethoxy)benzonitrile or, particularly, 4-[(2S)-oxiranylmethoxy]benzonitrile.

Unless otherwise stated, when molar equivalents and stoichiometric ratios are quoted herein with respect to acids and bases, these assume the use of acids and bases that provide or accept only one mole of hydrogen ions per mole of acid or base, respectively. The use of acids and bases having the ability to donate or accept more than one mole of hydrogen ions is contemplated and requires corresponding recalculation of the quoted molar equivalents and stoichiometric ratios. Thus, for example, where the acid employed is diprotic, then only half the molar equivalents will be required compared to when a monoprotic acid is employed. Similarly, the use of a dibasic compound (e.g. Na₂CO₃) requires only half the molar quantity of base to be employed compared to what is necessary where a monobasic compound (e.g. NaHCO₃) is used, and so on.

The skilled person will appreciate that certain compounds of formula IX may be prepared from certain other compounds of formula IX, or from structurally related compounds. For example, compounds of formula IX in which R¹ represents certain structural fragments of formula IXa may be prepared, in accordance with relevant processes known in the art, by the interconversion of corresponding compounds of formula IX in which R¹ represents different structural fragments of formula IXa (for example by analogy with the processes described in international patent application numbers WO 99/31100, WO 00/76997, WO 00/76998, WO 00/76999, WO 00/77000 and WO 01/28992).

It will be appreciated by those skilled in the art that, in the processes described above, the functional groups of reagents may be, or may need to be, protected by protecting groups.

In any event, functional groups which it is desirable to protect include hydroxy and amino. Suitable protecting groups for hydroxy include trialkylsilyl and diarylalkyl-silyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include the amino protective groups mentioned hereinbefore, such as benzyl, sulfonyl (e.g. benzenesulfonyl or 4-nitrobenzene-sulfonyl), tert-butyloxycarbonyl, 9-fluorenylmethoxycarbonyl or benzyloxycarbonyl.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

The process according to the first aspect of the invention may have the advantage that the salt of formula I is produced via a method that utilises fewer reagents than the processes of WO 02/083690 and fewer solvents than the processes of WO 2004/035592. Furthermore, process has the additional advantage that it is capable of providing the salt of formula I in a form (i.e. as a solution in a solvent system comprising water and a C₃₋₅ secondary alkyl alcohol) that is more convenient for subsequent manipulation to compounds of formula IX.

The process according to the second aspect of the invention may have the advantage that the compounds of formula IX, are, compared to the processes described in WO 02/083690 and WO 2004/035592, prepared in higher yields and by way by way of processes that comprise fewer steps and utilise fewer reagents and solvents.

In any event, the processes according to the invention may have the advantage that the salts of formula I, or the compounds of formula IX, are prepared in higher yields, in higher purity, by way of fewer steps, in less time, in a more convenient manner, in a more convenient form (e.g. in a form that is easier to handle), from more convenient (e.g. easy to handle) precursors, at a lower cost and/or with less usage and/or wastage of materials (including reagents and solvents) compared to the procedures disclosed in the prior art.

The term “relative volume” (rel. vol.), when used herein, refers to the volume (in millilitres) per gram of reagent employed.

“Substantially”, when used herein, may mean at least greater than 50%, preferably greater than 75%, for example greater then 95%, and particularly greater than 99%.

The invention is illustrated, but in no way limited, by the following examples.

### Synthesis of Intermediates

The following intermediates were not commercially available, and were therefore prepared by the methods described below.

### Preparation A

### 4-(2-Bromoethoxy)-3-fluorobenzonitrile

### (i) 4-Bromo-2-fluorophenol

Bromine (68.7 mL, 1.339 mol) dissolved in acetic acid (300 mL) was added drop by drop to a cooled solution of 2-fluorophenol (150 g, 1.339 mol) in acetic acid (1300 mL). The resulting mixture was stirred at room temperature overnight before being quenched with aqueous sodium bisulfite solution and extracted with dichloromethane. The organic layer was washed with water and brine and then dried over sodium sulfate. Solvent evaporation under reduced pressure afforded 4-bromo-2-fluorophenol (210 g) as a liquid. This was employed directly in the next step without further purification.

### (ii) 4-Bromo-2-fluoro-1-methoxybenzene

Methyl iodide (182.1 mL, 1.319 mol) was added at 0° C. to a well stirred suspension of 4-bromo-2-fluorophenol (210 g, 1.099 mol; see step (i) above) and K₂CO₃ (303.92 g, 2.19 mol) in dry acetone (1.7 L). Stirring was continued at 60° C. for two days under a nitrogen atmosphere before the reaction mixture was filtered and the solvent was concentrated under reduced pressure. This provided 4-bromo-2-fluoro-1-methoxybenzene (225 g) as a liquid, which was employed directly in the next step without further purification.

### (iii) 3-Fluoro-4-methoxybenzonitrile

A mixture of 4-bromo-2-fluoro-1-methoxybenzene (107 g, 0.52 mol; see step (ii) above), CuCN (70.4 g, 0.78 mol) in dry DMF (150 mL) was stirred at 120° C. overnight. The reaction mixture was cooled to room temperature, diluted with water and extracted with ethyl acetate. The organic layer was washed with water and brine and then dried over sodium sulfate. Solvent evaporation under reduced pressure, followed by column chromatography over silica gel using 3% ethyl acetate in petroleum ether as eluent, gave 24.4 g of the sub-title compound as a solid.

### (iv) 3-Fluoro-4-hydroxybenzonitrile

BBr₃ (23 mL, 0.242 mol) was added to 3-fluoro-4-methoxy-benzonitrile (24.4 g, 0.16 mol; see step (iii) above) in dichloromethane (200 mL) at −78° C. Stirring was continued at room temperature overnight. Another portion of BBr₃ (23 mL, 0.242 mol) was added at −78° C. and stirring was continued at RT for a further 2 days under a nitrogen atmosphere. The reaction mixture was quenched with ice water and extracted with dichloromethane. The organic layer was washed with water and brine, and then dried over sodium sulfate. Solvent evaporation under reduced pressure gave 20 g of the sub-title compound as a solid. This was employed directly in the next step without further purification.

### (v) 4-(2-Bromoethoxy)-3-fluorobenzonitrile

A suspension of 3-fluoro-4-hydroxybenzonitrile (20 g, 0.1459 mol; see step (iv) above), anhydrous K₂CO₃ (40.33 g, 0.2918 mol) and 1,2-dibromoethane (76.8 mL, 0.8754 mol) in dry DMF (150 mL) was stirred at 60° C. for 5 days under a nitrogen atmosphere. The reaction mixture was filtered through Celite and the solvent was evaporated under reduced pressure. The residue was purified by column chromatography over silica gel, using 2% ethyl acetate in petroleum ether as eluent, to yield 21.6 g of the title compound as a solid.

### Preparation B

### 2-(4-Cyano-2-fluorophenoxy)ethyl toluene-4-sulfonate

### Alternative I

### (i) 3-Fluoro-4-(2-hydroxyethoxy)benzonitrile

To potassium tert-butoxide (19.35 g) was added ethylene glycol (160 mL). The mixture was then heated to 50° C. At 50° C., 3,4-difluorobenzonitrile (20 g) was added and this was washed in with ethylene glycol (40 mL). The combined solution was heated to 80° C., and held at this temperature for two hours, before being cooled to 20° C. over one hour. The reaction mixture was filtered and washed with ethylene glycol (40 mL). To the filtrate was added water (200 mL) and dichloromethane (200 mL). The layers were separated and the organic layer was concentrated in vacuo, to give the sub-title compound as a waxy white solid (26.1 g, 100% yield).

¹H-NMR (CDCl₃, 300 MHz) δ 7.48-7.34 (m, 2H, CHₐᵣ), 7.05 (t, J=8.3 Hz, 1H, CH, 4.21 (t, J=4.5 Hz, 2H, CH₂), 4.08-3.98 (m, 2H, CH₂).

If necessary, 3-fluoro-4-(2-hydroxyethoxy)benzonitrile can be recrystallised using the following procedure.

To 3-fluoro-4-(2-hydroxyethoxy)benzonitrile (4.0 g) was added toluene (20 mL), and this mixture was heated to 65° C. At 65° C., all of the material had dissolved. The mixture was allowed to cool to room temperature (approximately 20° C.). Crystallisation was noticed at between 45 and 40° C. The reaction mixture was further cooled to 5° C. The reaction mixture was filtered and was washed with toluene (5 mL). The damp solid was dried in vacuo, at 35° C., to give the purified sub-title compound as an off-white, crystalline solid (3.38 g; 85% yield).

¹H-NMR (CDCl₃, 300 MHz) δ 7.46-7.34 (m, 2H, CHₐᵣ), 7.04 (t, J=8.3 Hz, 1H, CHₐᵣCFₐᵣ), 4.21 (t, J=4.5 Hz, 2H, CₐᵣOCH₂), 4.03 (q, J=5.1 Hz, 2H, CH₂OH), 2.09 (t, J=6.3 Hz, 1H, OH).

### (ii) 2-(4-Cyano-2-fluorophenoxy)ethyl toluene-4-sulfonate

To 3-fluoro-4-(2-hydroxyethoxy)benzonitrile (47.6 g; see step (i) above) was added dichloromethane (380 mL). To this was added triethylamine (55 mL) and then, over approximately sixty minutes, a solution of para-toluenesulfonyl chloride (50 g) dissolved in dichloromethane (380 mL). Water (380 mL) was added to the resulting mixture and the layers were separated. The lower (organic) layer was concentrated in vacuo to give the title compound as a white solid (87.9 g; 99.8%).

Recrystallisation of the title compound can be carried out, if necessary, using any of the methods below.

### Method 1

To 2-(4-cyano-2-fluorophenoxy)ethyl toluene-4-sulfonate (167.7 g) was added ethyl acetate (1.65 L). This mixture was then heated to reflux (approximately 78° C.), at which point all of the material had dissolved. The reaction mixture was allowed to cool to room temperature (approximately 20° C.). Crystallisation was noticed at between 70° C. and 75° C. The reaction mixture was cooled to 5° C. The mixture was filtered, and was washed with ethyl acetate (165 mL). The damp solid was dried in vacuo, at 35° C., to give purified title compound as a white, crystalline solid (103.3 g; 61.6%).

### Method 2

To 2-(4-cyano-2-fluorophenoxy)ethyl toluene-4-sulfonate (10 g) was added toluene (75 mL) and acetonitrile (5 mL). This mixture was heated to 80° C. At 80° C., all of the material had dissolved. The reaction mixture was cooled to room temperature (approximately 20° C.). Crystallisation was noticed at between 55 and 50° C. The reaction mixture was further cooled to 5° C. The mixture was filtered, and the solid was washed with toluene (10 mL). The damp solid was dried in vacuo, at 35° C., for approximately eighteen hours, to give purified title compound as an off-white crystalline solid (9 g, 90% yield).

### Method 3

To 2-(4-cyano-2-fluorophenoxy)ethyl toluene-4-sulfonate (10 g) was added toluene (75 mL). This mixture was heated to 95° C. At 95° C., all of the material had dissolved. The reaction mixture was cooled to room temperature (approximately 20° C.). Crystallisation was noticed at between 65 and 60° C. The reaction mixture was further cooled to 5° C. The mixture was filtered, and the solid was washed with toluene (10 mL). The damp solid was dried in vacuo, at 35° C., for approximately seventeen hours, to give purified title compound as an off-white crystalline solid (9.4 g, 94% yield).

### Alternative II

To 3-fluoro-4-hydroxybenzonitrile (0.2 kg) was added acetonitrile (0.85 L), at 20° C. To this was added potassium carbonate (404 g); this was washed in with acetonitrile (0.18 L). The reaction was then heated to 80° C.±5° C., at approximately 1° C. per minute. When the reaction mixture was at 80° C.±5° C., 2-bromoethan-1-ol (0.31 L) was added, over approximately twenty minutes. This was washed in with acetonitrile (0.18 L). The temperature was adjusted to 80° C.±5° C., and maintained at this temperature for six hours. The reaction mixture was then cooled to 30° C., at approximately 1° C. per minute. For convenience, the reaction was held at 30° C. for approximately 12 hours. Toluene (1.6 L) and water (1.34 L) were then added to the reaction mixture. The reaction mixture was re-heated to 30° C. The layers were separated, and the lower (aqueous) layer (approximately 1.2 L) was discarded. The upper (organic) layer was distilled at reduced pressure to remove approximately six volumes of solvent (approximately 1.2 L at less than 55° C.). The reaction mixture was then cooled to 20° C., and was analysed for water content (typically <0.1% w/w). To this was added triethylamine (245 mL), and the reaction mixture was cooled to −10° C. To this was added trimethylamine hydrochloride (28 g), followed by a solution of para-toluenesulfonyl chloride (292 g) dissolved in toluene (1.2 L), whilst maintaining the temperature at −10° C.±10° C. When the addition was complete the reaction mixture was warmed to 20° C. To this was added water (1.2 L), and the reaction mixture was heated to 75° C. At 75° C., the layers were separated, and the lower (aqueous) layer was discarded. To the retained organic layer was added 1 M hydrochloric acid (1.2 L), the reaction mixture was heated to 80° C. The layers were separated and the lower (aqueous) layer was discarded. The upper (organic) layer was cooled to 20° C. over approximately two hours. For convenience, the reaction mixture was held at 20° C. for approximately twelve hours. The reaction mixture was then cooled to 5° C. over approximately thirty minutes. The reaction mixture was held at 5° C. for approximately one hour. The mixture was filtered, and the crude solid was then washed with toluene (200 mL, 5° C.). The damp solid was dried in vacuo, at 35° C., for approximately twenty-four hours, to give the title compound as a white, crystalline solid (373 g, 76% yield).

¹H-NMR (CDCl₃, 300 MHz) δ 7.80 (d, J=8.4 Hz, 2H, CHₐᵣ), 7.41-7.32 (m, 4H, CHₐᵣₛ), 6.94 (d, J=8.2 Hz, 1H, CHₐᵣ), 4.44-4.38 (m, 2H, CH₂), 4.34-4.28 (m, 2H, CH₂), 2.45 (s, 3H, ArCH₃).

### Preparation C

### [3-(7-Benzyl-9-oxa-3,7-diazabicyclo[3.3.1]non-3-yl)propyl]carbamic acid tert-butyl ester, 4-chlorobenzenesulfonic acid salt

To 3-bromopropylamine hydrobromide (139.32 g, 636.40 mmol) was added a solution of di-tent-butyl dicarbonate (112.46 g, 510.13 mmol) in MIBK (800 mL) and 2.5 M sodium hydroxide (310 mL). The resulting mixture was stirred for 1 hour at room temperature. The reaction was monitored by TLC (9:1 isohexane:ethyl acetate, potassium permanganate stain). Water (345 mL) was added and the mixture stirred for 10 minutes. The phases were separated and the lower (aqueous) phase discarded. To the retained organic phase was added 3-benzyl-9-oxa-3,7-diazabicyclo[3.3.1]nonane dihydrochloride (148.43 g, 509.68 mmol; see WO 02/083690) and 2.5 M sodium hydroxide (660 mL). This mixture was heated at 65° C. for 7 hours. At 65° C., the phases were separated and the lower (aqueous) phase discarded. The organic phase was re-heated to 65° C. and extracted with 10% w/w aqueous citric acid (562 mL). The phases were separated and the upper (organic) phase discarded. To the resulting aqueous phase was added MIBK (800 mL) and 5 M sodium hydroxide (230 mL) containing approximately 10% w/v sodium chloride (22.84 g). The resulting mixture was stirred at room temperature for 15 minutes. The phases were separated and the lower (aqueous) phase discarded. The organic phase was azeo-dried by removal of solvent (300 mL) by distillation under reduced pressure (keeping the temperature below 70° C.). The mixture was clarified by filtration whilst still hot and the residue washed with MIBK (115 mL). The temperature was adjusted to 60° C. and a solution of purified (see J. Am. Pharm. Assoc. 239-241 (1949)) 4-chlorobenzenesulfonic acid (99.24 g, 515.20 mmol) in MIBK (225 mL) was added over 90 minutes. The reaction mixture was then cooled to room temperature causing the product to crystallize from solution. The mixture was cooled to 5° C., the product was collected by filtration and the cake washed with MIBK (225 mL). The product was dried as far as possible on the filter, then oven dried in vacuo (50° C., 24 h) to give the title compound as a white solid (257.44 g, 453.13 mmol, 89%).

¹H NMR (300 MHz, DMSO-d₆) δ 7.61 (d, J=8.7 Hz, 2H), 7.46-7.35 (m, 7H), 7.10 (t, J=5.7 Hz, 1H), 4.15 (s, 2H), 3.70 (s, 2H), 3.40 (d, J=12.1 Hz, 3H), 3.07 (d, J=11.9 Hz, 4H), 2.97 (q, J=6.3 Hz, 2H), 2.84 (t, J=7.1 Hz, 2H), 2.76 (d, J=11.9 Hz, 2H), 1.70 (quintet, J=6.7 Hz, 2H), 1.45 (s, 9H).

### Preparation D

### 3-(4-Cyanophenoxypropyl) 4-methylbenzenesulfonate

### Alternative I

### (i) 4-(3-Hydroxypropoxy)benzonitrile

To a flask was added 4-hydroxybenzonitrile (50 g, 0.41 mol, 1 eq.) and potassium carbonate (0.51 mol, 1.25 eq.). To this mixture was added 4-methyl-2-pentanone (400 mL). Stirring was started and 3-bromo-1-propanol (61.50 g, 0.4 mol, 1.05 eq.) was added in one portion. The reaction mixture was heated to between 85 and 90° C. for 5 hours. Water (250 mL) was then added and the resultant mixture heated to 30° C. until all solids had gone into solution. The aqueous layer was separated from the organic layer. The organic layer was diluted with 4-methyl-2-pentanone (400 mL) to provide a solution of the sub-title compound that was employed directly in the next step without further purification.

GC: 95% pure, LC: 96.50%

GC-MS: m/z=177.

¹H NMR (300 MHz, CDCl₃) δ 1.50 (t, J=5.7 Hz, 1H), 2.07 (quintet, J=6.0 Hz, 2H), 3.87 (q, J=5.7 Hz, 2H), 4.17 (t, J=6.0 Hz, 2H), 6.96 (dd, J=6.9, 2.1 Hz, 2H), 7.59 (dd, J=6.9, 2.1 Hz, 2H).

### (ii) 3-(4-Cyanophenoxypropyl) 4-methylbenzenesulfonate

The solution generated in step (i) above was distilled under reduced pressure (distillate temperature 50° C. and pressure 100 mbar). About 500 mL of the solvent was distilled off. The water content of the residue was about 0.002% w/w. The residue was diluted with 4-methyl-2-pentanone (400 mL) and triethylamine (53.70 g, 0.53 mol, 1.25 eq.) added. The reaction mixture was cooled to −15° C. and trimethylamine hydrochloride (8.16 g, 0.083 mol, 0.2 eq.) added. To the stirring solution was added p-toluenesulfonyl chloride (85.80 g, 0.445 mol, 1 eq.) in 4-methyl-2-pentanone (400 mL), whilst keeping the temperature below −10° C. The reaction mixture was stirred at below −10° C. for 3 hours before being allowed to warm slowly to room temperature, at which temperature stirring was continued for a further 18 hours. Water (300 mL) was added to the reaction mixture and the resulting slurry was heated (ca. 85° C.) until all the solids had gone into solution. The aqueous layer was separated from the organic layer. To the organic layer was added hydrochloric acid (200 mL, 1M). The resulting mixture was then heated (ca. 85° C.) until all the solids were in solution. The aqueous layer was separated from the organic layer. The organic layer was allowed to come to room temperature and was then cooled (5° C.) for 2 hours. The precipitated solid was isolated by filtration and washed with 4-methyl-2-pentanone (100 mL) before being dried in an oven at 50° C. under reduced pressure. This yielded the title compound as a colourless solid (114.25 g, 82%).

¹H-NMR (300 MHz, CDCl₃,) δ 2.11-2.19 (2H, m), 3.99-4.04 (2H, t), 4.22-4.26 (2H, t), 6.81-6.84 (2H, m), 7.25-7.26 (2H, m), 7.54-7.58 (2H, m), 7.74-7.77 (2H, m).

LC 98.7%.

(M+H+acetonitrile)⁺=373.

### Alternative II

### (i) 4-(3-Hydroxypropoxy)benzonitrile

To a flask was added 4-hydroxybenzonitrile (50 g, 0.41 mol, 1 eq.) and toluene (400 mL). The resulting mixture was heated to 65° C.±5° C. To the stirring reaction mixture was added 3-bromo-1-propanol (72.90 g, 0.51 mol, 1.25 eq.) and then, over the course of 20 minutes, sodium hydroxide (210 mL, 2.5 M, 0.52 mol, 1.25 eq.). The reaction was heated to 65 to 70° C. for 17 hours. The aqueous layer was separated from the organic layer at 60 to 65° C. The organic layer was then employed directly in the next step without further purification.

LC purity 95.3%.

¹H NMR (300 MHz, CDCl₃) δ 1.50 (t, J=5.7 Hz, 1H), 2.07 (quintet, J=6.0 Hz, 2H), 3.87 (q, J=5.7 Hz, 2H), 4.17 (t, J=6.0 Hz, 2H), 6.96 (dd, J=6.9, 2.1 Hz, 2H), 7.59 (dd, J=6.9, 2.1 Hz, 2H).

### (ii) 3-(4-Cyanophenoxypropyl) 4-methylbenzenesulfonate

Toluene (400 mL) was added to the solution generated in the previous step. About 330 mL of the solvent was distilled off under reduced pressure (at 50° C.). To the residue was added toluene (200 mL) and triethylamine (53.70 g, 0.53 mol, 1.25 eq.). The reaction mixture was cooled to −15° C. and trimethylamine hydrochloride (8.16 g, 0.083 mol, 0.2 eq.) added. To the stirring solution was added p-toluenesulfonyl chloride (85.80 g, 0.445 mol, 1 eq.) in toluene (300 mL), whilst keeping the temperature below −10° C. The residual p-toluenesulfonyl chloride was washed into the reaction mixture with toluene (100 mL). The reaction mixture was stirred at below −10° C. for 3 hours. The reaction mixture was allowed to warm to room temperature slowly and was then stirred for 18 hours. To the reaction mixture added water (300 mL) and resulting the slurry was heated (ca. 85° C.) until all the solids were in solution. The aqueous layer was separated from the organic layer. To the organic layer was added hydrochloric acid (200 mL, 1 M). The organic layer was allowed to cool to room temperature, and then to ca. 5° C., at which temperature it was stirred for 2 hours. The precipitated solid was isolated by filtration and then washed with toluene (100 mL). The product was dried in an oven (at 50° C.) under reduced pressure to yield the title compound as a colourless solid (94.20 g, 67%).

LC purity 99.1%

(M+H+acetonitrile)⁺=373.

¹H NMR (300 MHz, CDCl₃) δ 2.15 (quintet, J=5.9 Hz, 2H), 2.43 (s, 3H), 4.01 (t, J=5.9 Hz, 2H), 4.24 (t, J=5.9 Hz, 2H), 6.83 (dd, J=6.9, 1.9 Hz, 2H), 7.26 (t, J=3.9 Hz, 6H), 7.56 (t, J=16.2 Hz, 2H), 7.75 (d, J=8.2 Hz, 2H).

### Alternative III

### (i) 4-(3-Hydroxypropoxy)benzonitrile

To a flask was added 4-hydroxybenzonitrile (10 g, 82.7 mmol, 1 eq.) and potassium carbonate (13.60 g, 98.7 mmol, 1.25 eq.). To this mixture was added acetonitrile (80 mL) and then, under stirring, 3-bromo-1-propanol (12.25 g, 86.40 mmol, 1.05 eq.). The reaction mixture was heated at reflux (84° C.) for 5 hours before being allowed to cool to room temperature. Toluene (80 mL) and water (50 mL) were added and the resulting mixture was heated (≈30° C.) until all of the solids had gone into solution. The aqueous layer was separated from the organic layer. The organic layer was retained.

¹H-NMR (300 MHz, CDCl₃) δ 1.50 (t, J=5.7 Hz, 1H), 2.07 (quintet, J=6.0 Hz, 2H), 3.87 (q, J=5.7 Hz, 2H), 4.17 (t, J=6.0 Hz, 2H), 6.96 (dd, J=6.9, 2.1 Hz, 2H), 7.59 (dd, J=6.9, 2.1 Hz, 2H).

### (ii) 3-(4-Cyanophenoxypropyl) 4-methylbenzenesulfonate

The solution generated in step (i) above was distilled to remove 40 mL of the solvent. The mixture was then allowed to cool to room temperature before triethylamine (10.09 g, 98.7 mmol, 1.25 eq.) was added. The reaction mixture was cooled to −15° C. and trimethylamine hydrochloride (1.57 g, 16.45 mmol, 0.2 eq.) was added. To the stirring reaction mixture was added p-toluenesulfonyl chloride (16.47 g, 86.38 mmol, 1.05 eq.) in toluene (60 mL), whilst keeping the temperature below −10° C. The reaction mixture was stirred at below −10° C. for 3 hours before being allowed to warm to room temperature. Water (60 mL) was added and the resulting slurry was heated to ca. 60° C. until all the solids had gone into solution. The aqueous layer was separated from the organic layer and hydrochloric acid (60 mL, 0.5 M) was added to the organic layer. The resulting mixture was heated to ca. 62° C. until all the solids were in solution. The organic layer was separated from the aqueous layer, was allowed to cool to room temperature and was then stirred at ca. 5° C. for 2 hours. The precipitated solid was isolated by filtration and washed with toluene (20 mL). The product was dried in an oven (at 40° C.) under reduced pressure to yield the title compound as a colourless solid (19.92 g, 73%).

¹H-NMR (300 MHz, CDCl₃,) δ 2.11-2.19 (2H, m), 3.99-4.04 (2H, t), 4.22-4.26 (2H, t), 6.81-6.84 (2H, m), 7.25-7.26 (2H, m), 7.54-7.58 (2H, m), 7.74-7.77 (2H, m).

LC 99.6%.

(M+H+acetonitrile)⁺=373.

### EXAMPLES

### Example 1

### tert-Butyl 2-{7-[(2S)-3-(4-cyanophenoxy)-2-hydroxypropyl]-9-oxa-3,7-diaza-bicyclo[3.3.1]non-3-yl}ethylcarbamate

### Alternative 1

### (a)[2-9-Oxa-3,7-diazabicyclo[3.3.1]non-3-yl)ethyl]carbamic acid tert-butyl ester, 2,4,6-trimethylbenzenesulfonic acid salt

[2-(7-Benzyl-9-oxa-3,7-diazabicyclo[3.3.1]non-3-yl)ethyl]carbamic acid tert-butyl ester 2,4,6-trimethylbenzenesulfonic acid salt (150 g; see, for example, WO 2004/035592), isopropanol (IPA; 450 mL) and water (150 mL) were combined in a metal hydrogenation vessel. Solid 5% Pd/C catalyst (4.5 g, 61% water wet, Johnson Matthey type 440) was added. The mixture was then hydrogenated under 2.5 bar of hydrogen pressure and was simultaneously heated to 55° C. Gas uptake measurement showed the reaction to be complete after 1 hour. After cooling to 39° C. the catalyst was removed by filtration through a glass fibre filter paper. The catalyst was washed on the filter with IPA (150 mL) and the combined filtrate and washings used in the next step.

### (b) tent-Butyl 2-{7-[(2S)-3-(4-cyanophenoxy)-2-hydroxypropyl]-9-oxa-3,7-diaza-bicyclo[3.3.1]non-3-yl}ethylcarbamate

Aqueous sodium carbonate solution (1 M, 133 mL) was added to a solution of [2-(9-oxa-3,7-diazabicyclo[3.3.1]non-3-yl)ethyl]carbamic acid tert-butyl ester, 2,4,6-trimethylbenzenesulfonic acid salt (see step (a) above). A solution of 4-[(2S)-oxiranylmethoxy]benzonitrile (44.4 g; see, for example WO 01/28992) in IPA (75 mL) and toluene (75 mL) was added. The reaction was heated to 73° C. for 4 hours and then left to stir at ambient temperature overnight. Solvent (440 mL) was removed by distillation at less than 84° C. Toluene (1 L) was added and solvent was distilled (water 52 mL, organic solvent 441 mL). A further portion of toluene was added (500 mL) and solvent was distilled again (water 82 mL, organic solvent 437 mL). The mixture was then cooled to ambient temperature. Aqueous sodium hydroxide (1 M, 450 mL) was added and the mixture was stirred for 5 minutes and then the phases were separated. The aqueous phase was discarded and the toluene phase washed with aqueous citric acid (10% w/v, 450 mL). The toluene phase was discarded. 4-Methyl-2-pentanol (MIBC; 600 mL) and aqueous sodium hydroxide (5 M, 450 mL) were added to the citric acid phase. After stirring for 5 minutes the phases were separated and the aqueous discarded. The MIBC phase was washed with aqueous sodium chloride (20% w/v, 150 mL). The mixture of MIBC and aqueous sodium chloride was concentrated under reduced pressure at less than 50° C. (water (20 mL) and MIBC (55 mL) was collected). The MIBC solution was cooled to 33° C. and then left to stir overnight. The solution was filtered to a clean vessel. Solvent (285 mL) was distilled under reduced pressure at less than 70° C. Diisopropyl ether (IPE; 900 mL) was added at such a rate so that the temperature remained above 55° C. The solution was then allowed to cool to 23° C. After 90 minutes, crystallisation started and the mixture was stirred for 15 minutes before being cooled to 5° C. The product was collected by filtration. The solid was washed on the filter with IPE (300 mL) and sucked dry. Further drying in vacuo at 55° C. gave the title compound as a white solid (92.5 g, 78% over two steps).

### Alternative 2

### (a) [2-(9-Oxa-3,7-diazabicyclo[3.3.1]non-3-yl)ethyl]carbamic acid tert-butyl ester, 2,4,6-trimethylbenzenesulfonic acid salt

[2-(7-Benzyl-9-oxa-3,7-diazabicyclo[3.3.1]non-3-yl)ethyl]carbamic acid tert-butyl ester 2,4,6-trimethylbenzenesulfonic acid salt (150 g; see, for example, WO 2004/035592), isopropanol (IPA; 225 mL) and water (75 mL) were combined in a metal hydrogenation vessel. Solid 5% Pd/C catalyst (4.7 g, 61% water wet, Johnson Matthey type 440) was added. Hydrogen was introduced to the vessel and stirring was started. The mixture was hydrogenated under 2.5 bar of hydrogen pressure and was simultaneously heated to 55° C. (temperature overshot to 73° C.). Gas uptake measurement showed the reaction to be complete after 1 hour. After cooling to 47° C. the catalyst was removed by filtration through a glass fibre filter paper. The catalyst was washed on the filter with IPA (75 mL) and the combined filtrate and washings used in the next step.

### (b) tert-Butyl 2-{7-[(26)-3-(4-cyanophenoxy)-2-hydroxypropyl]-9-oxa-3,7-diaza-bicyclo[3.3.1]non-3-yl}ethylcarbamate

A solution of [2-(9-oxa-3,7-diazabicyclo[3.3.1]non-3-yl)ethyl]carbamic acid tert-butyl ester, 2,4,6-trimethylbenzenesulfonic acid salt (see step (a) above) was warmed to 55° C. Aqueous sodium carbonate solution (1 M, 133 mL) was added, followed by a warm (40° C.) solution of 4-[(2S)-oxiranylmethoxy]benzonitrile (44.4 g; see, for example WO 01/28992) in IPA (75 mL) and toluene (75 mL). The solution was rinsed into the reaction flask with IPA (37 mL) and toluene (37 mL). The reaction was heated to 78° C. for 4 hours and then left to stir at ambient temperature overnight. Toluene was added (1050 mL) and solvent was distilled (600 mL). The mixture was then allowed to cool to 26° C. Aqueous sodium hydroxide (1 M, 450 mL) was added. The mixture was stirred for 5 minutes and then the phases were separated. The aqueous phase was discarded and the toluene phase washed with aqueous citric acid (10% w/v, 450 mL). The toluene phase was discarded. 4-Methyl-2-pentanol (MIBC; 600 mL) and aqueous sodium hydroxide (5 M, 450 mL) were added to the citric acid phase. After stirring for 5 minutes the phases were separated and the aqueous phase discarded. The MIBC phase was washed with aqueous sodium chloride (20% w/v, 150 mL) and the phases separated. The MIBC solution was then left to stir overnight (this overnight stir is unnecessary but in this example was carried out for convenience). The MIBC phase was concentrated under reduced pressure (78 mL of solvent was collected). The solution was filtered to a clean vessel, washing through with MIBC (150 mL). Solvent (437 mL) was distilled under reduced pressure at <70° C. Diisopropyl ether (IPE; 900 mL) was added at 55° C. and the temperature fell to 40° C. The solution was re-heated to 58° C. and then allowed to cool naturally to ambient temperature (at 28° C. a precipitate forms). The mixture was stirred overnight at ambient temperature. The mixture was cooled to 5° C. and the solid collected by filtration. The filter cake was washed by displacement with IPE (300 mL) and dried by suction on the filter. Further drying in vacuo at 70° C. gave the title compound as a white solid (97.3 g, 82% over two steps).

### Alternative 3

### (a) [2-(9-Oxa-3,7-diazabicyclo[3.3.1]non-3-yl)ethyl]carbamic acid tert-butyl ester, 2,4,6-trimethylbenzenesulfonic acid salt

[2-(7-Benzyl-9-oxa-3,7-diazabicyclo[3.3.1]non-3-yl)ethyl]carbamic acid tert-butyl ester 2,4,6-trimethylbenzenesulfonic acid salt (100 g of material that was 3.5% w/w water; see, for example, WO 2004/035592) was added to a metal hydrogenation vessel. Pre-mixed isopropanol (IPA; 150 mL) and water (50 mL) was added. Solid 5% Pd/C catalyst (4.0 g, 61% water wet, Johnson Matthey type 440) was added. Hydrogen was introduced to the vessel and stirring was started. The mixture was hydrogenated under 3.5 bar of hydrogen pressure and was simultaneously heated to 55° C. (temperature overshot to 68° C.). Gas uptake measurement showed the reaction to be complete after 3.5 hours. The reaction was filtered directly to the next reaction vessel at the appropriate point detailed below. The catalyst was washed with IPA (50 mL) and the wash added directly to the next reaction vessel at the appropriate point detailed below.

### (b) tert-Butyl 2-{7-[(2S)-3-(4-cyanophenoxy)-2-hydroxypropyl]-9-oxa-3,7-diaza-bicyclo[3.3.1]non-3-yl}ethylcarbamate

A clean vessel was charged with 4-[(2S)-oxiranylmethoxy]benzonitrile (30.1 g; see, for example WO 01/28992), followed by an aqueous solution of sodium carbonate (0.3 M, 300 mL). A solution of [2-(9-oxa-3,7-diazabicyclo[3.3.1]non-3-yl)ethyl]carbamic acid tert-butyl ester, 2,4,6-trimethylbenzenesulfonic acid salt (see step (a) above) was added followed by the catalyst wash (see step (a) above). The mixture was heated at reflux (78° C.) for 4 hours and then left at ambient temperature for 4 days (this standing period is unnecessary but in this example was carried out for convenience). Solvent was removed (236 mL) by distillation under reduced pressure (approximately 2.5 volumes of solvent need to be distilled to ensure removal of IPA). Toluene (400 mL) and aqueous sodium hydroxide (3 M, 100 mL) were added and the mixture stirred for 5 minutes. The phases were separated at 27° C. and the lower aqueous phase discarded. Aqueous citric acid (10% w/v, 300 mL) was added to the remaining toluene phase. After stirring for 5 minutes the phases were separated and the upper toluene phase discarded. 4-Methyl-2-pentanol (MIBC; 600 mL) and an aqueous solution of sodium hydroxide (5 M, 450 mL) containing sodium chloride (at 10% w/v) were added to the citric acid phase. After stirring for 5 minutes the phases were separated at 30° C. and the aqueous phase discarded. The MIBC phase was washed with aqueous sodium chloride (20% w/v, 100 mL) and after 5 minutes stirring the phases separated. The MIBC solution was then left to stand overnight (this overnight stand is unnecessary but in this example was carried out for convenience). The MIBC phase was concentrated under vacuum at a temperature of less than 44° C. (maximum temperature that can be reached at this part of the process is 70° C.); solvent was collected (water 18 mL: MIBC 35 mL). The solution was filtered to a clean vessel, washing through with MIBC (50 mL). Solvent (240 mL) was distilled under vacuum at less than 70° C. Diisoprepyl ether (IPE; 600 mL) was added and the solution was re-heated to 64° C. The solution was stirred at 250 rpm and allowed to cool naturally. After 2 hours stirring, the temperature had fallen to 28° C. and precipitation of product had started. After stirring for a further 90 minutes, the temperature had fallen to 21° C. The mixture was cooled to 5° C. in 20 minutes and then held at this temperature for 90 minutes. The product was collected by filtration. The filter cake was washed by displacement with IPE (200 mL; IPE temperature was 20° C.) and dried by suction on the filter. The product was dried overnight in vacuo at 35° C. to give the title compound as a white solid (65.2 g, 85% over two steps).

### Alternative 4

### (a) [2-(9-Oxa-3,7-diazabicyclo[3.3.1]non-3-yl)ethyl]carbamic acid tert-butyl ester, 2,4,6-trimethylbenzenesulfonic acid salt

[2-(7-Benzyl-9-oxa-3,7-diazabicyclo[3.3.1]non-3-yl)-ethyl]carbamic acid tert-butyl ester 2,4,6-trimethylbenzenesulfonic acid salt (92.60 kg of material that was 17.51% w/w water; see, for example, WO 2004/035592) and solid 5% Pd/C catalyst (3.70 kg, 61% water wet, Johnson Matthey type 440) was added to a metal hydrogenation vessel. Pre-mixed isopropanol (IPA; 109.30 kg) and water (46.2 kg) was added. The vessel was purged with hydrogen to 0.5 bar to displace nitrogen and then hydrogen introduced to the vessel to 3.0 bar, stirring was started and simultaneous heating to 55° C. was begun (maximum temperature reached was 55.3° C.). The reaction mixture was held under hydrogen for 1 hour 45 minutes before the uptake of gas had stopped indicating that the reaction was complete. The reaction mixture was then cooled to 20° C. and left to stand for 21 hours 35 minutes (the standing period is unnecessary but was carried out for convenience). The reaction mixture was filtered into the next reaction vessel where indicated below and the catalyst cake was washed with IPA (35.9 kg) and added into the next reaction vessel where indicated below.

### (b) tert-Butyl 2-{7-[(2S)-3-(4-cyanophenoxy)-2-hydroxypropyl]-9-oxa-3,7-diaza-bicyclo[3.3.1]non-3-yl}ethylcarbamate

A clean vessel was charged with 4-[(2S)-oxiranylmethoxy]benzonitrile (22.50 kg; see, for example WO 01/28992), demineralised water (184.7 kg) and sodium carbonate solution (1 M, 91.2 kg). A solution of [2-(9-oxa-3,7-diazabicyclo-[3.3.1]non-3-yl)ethyl]carbamic acid tert-butyl ester, 2,4,6-trimethylbenzene-sulfonic acid salt (see step (a) above) was added and the catalyst wash (see step (a) above) was also added. The mixture was heated to 78° C. over 35 minutes and held at this temperature for 4 hours, then cooled to 25.1° C. and left at room temperature for 84 hours 42 minutes (this standing period is unnecessary in this example was carried out for convenience). Solvent (215.3 kg) was removed by distillation under reduced pressure. Toluene (321.0 kg) was added and temperature of the reaction mixture was adjusted to 25.5° C. Sodium hydroxide solution (3 M, 101.7 kg) was charged to the reaction vessel and stirred for 23 minutes. Agitation was stopped and the phases were allowed to separate over 30 minutes. The lower aqueous phase was discarded. Stirring of the organic upper phase was restarted and aqueous citric acid solution (10% w/w, 278.3 kg) was added and stirred for 23 minutes. The agitation was stopped and the phases were allowed to separate over 40 minutes. The lower aqueous phase was sent to a second vessel (VESSEL 2) and the upper organic phase was discarded. The aqueous phase was then returned to the reaction vessel, stirring started and 4-methyl-2-pentanol (MIBC; 297.7 kg) and a pre-mixed solution of sodium hydroxide (10 M, 185.4 kg) and sodium chloride solution (20% w/w, 111.1 kg) was added and stirred for 15 minutes. Agitation was then stopped and the phases were allowed to separate over 30 minutes. The lower aqueous phase was discarded. Agitation was restarted and sodium chloride solution (20% w/w, 111.1 kg) was added and the contents of the reaction vessel stirred for 10 minutes. Agitation was stopped and the phases were allowed to separate for 18 minutes. The lower aqueous phase was discarded. Stirring was started and solvent (42.3 kg) was removed from the retained upper organic phase by distillation under reduced pressure. The concentrated solution was transferred to a clean vessel (VESSEL 3) and the reaction vessel was washed with water to remove residual salt contamination. The organic phase was then heated to 47.3° C. and filtered hot into the cleaned reaction vessel. MIBC (37.3 kg) was added to VESSEL 3 and then filtered into the reaction vessel and combined with the bulk of the solution. Solvent (240.3 kg) was then removed by distillation under reduced pressure keeping the temperature of the mixture below 70° C., after which the temperature was adjusted to 53.1° C. and diisopropyl ether (313.9 kg) was added. The temperature was adjusted to 51.6° C. and then cooled to 20° C. over 110 minutes and left to stand for 14 hours 49 minutes (this standing period is unnecessary but was carried out for convenience). The slurry was then cooled to 5° C. over 30 minutes and held at 5° C. for 30 minutes. The mixture was then filtered and a displacement wash of cold (5° C.) diisopropyl ether (134.5 kg) was added and the cake was blown with nitrogen for 135 minutes (this is unnecessary but was carried out for convenience). The solid was then dried on the filter under reduced pressure with heating at 30° C. for 87 hours to give the title compound as a white solid (49.05 kg, 80.7%).

### Alternative 5

### (a) [2-(9-Oxa-3,7-diazabicyclo[3.3.1]non-3-yl)-ethyl]-carbamic acid tert-butyl ester, 2,4,6-trimethylbenzenesulfonic acid salt

[2-(7-Benzyl-9-oxa-3,7-diazabicyclo[3.3.1]non-3-yl)ethyl]carbamic acid tert-butyl ester, 2,4,6-trimethylbenzenesulfonic acid salt (150 g of material that was 3.33% w/w water; see, for example, WO 2004/035592) was added to a metal hydrogenation vessel. Pre-mixed isopropanol (IPA, 180 g) and water (75 g) was added. Solid 5% Pd/C catalyst (6.0 g, 61% water wet, Johnson Matthey type 440) was added. After nitrogen purge, hydrogen was introduced to the vessel and stirring was started. The mixture was hydrogenated under 3.5 bar of hydrogen pressure and was simultaneously heated to 65° C. over 15 minutes (temperature overshot to 73° C.). Gas uptake measurement showed the reaction to be complete after 30 minutes (which included the heat up time). After a further 30 minutes at 65° C., the reaction was cooled to 23° C. and then filtered directly into the next reaction vessel at the appropriate point detailed below. The catalyst was washed with IPA (60 g) and the wash added directly to the next reaction vessel at the appropriate point detailed below.

### (b) tert-Butyl 2-{7-[(2S)-3-(4-cyanophenoxy)-2-hydroxypropyl]-9-oxa-3,7-diaza-bicyclo[3.3.1]non-3-yl}ethylcarbamate

A clean vessel was charged with 4-[(2St)-oxiranylmethoxy]benzonitrile (44.3 g (see, for example WO 01/28992) 0.98 mol equiv. based on anhydrous [2-(7-benzyl-9-oxa-3,7-diazabicyclo[3.3.1]-non-3-yl)ethyl]carbamic acid tert-butyl ester, 2,4,6-trimethylbenzenesulfonic acid salt), followed by an aqueous solution of sodium carbonate (3% w/w, 480 g). The solution of [2-(9-oxa-3,7-diaza-bicyclo[3.3.1]non-3-yl)ethyl]carbamic acid tert-butyl ester, 2,4,6-trimethyl-benzenesulfonic acid salt (see step (a) above) was added, followed by the catalyst wash (see step (a) above). The resulting mixture was heated to reflux (78° C.) over 30 minutes and then held at this temperature for 2 hours. The reaction was cooled to 50° C. Solvent (353 g) was removed by distillation under reduced pressure at ≦50° C. Toluene (375 g) was added and the temperature adjusted to 28° C.±3° C. (All extraction operations that follow were conducted at this temperature). Aqueous sodium hydroxide (10% w/w, 180 g) was added and the mixture stirred for 5 minutes. The phases were separated and the lower aqueous phase discarded. Aqueous citric acid (10% w/w, 450 g) was added to the remaining toluene phase. After stirring for 5 minutes the phases were separated and the upper toluene phase discarded. 4-Methyl-2-pentanol (MIBC) (420 g) and an aqueous solution of sodium hydroxide/sodium chloride (15% w/w wrt NaOH, 7.5% w/w wrt NaCl, 600 g) were added to the citric acid phase. After stirring for 5 minutes the phases were separated and the aqueous phase discarded. The MIBC phase was washed with aqueous sodium chloride (20% w/w, 75 g) and after 5 minutes stirring the phases separated. The MIBC phase was concentrated under reduced pressure at ≦50° C. (84 g of solvent was removed). The solution was filtered to a clean vessel, washing through with MIBC (60 g). Solvent (239 g) was distilled out under vacuum at <70° C. Isopropyl ether (IPE) (653 g) was added and the solution was re-heated to above 55° C. The solution was stirred and allowed to cool overnight. The following day, the mixture was cooled from ambient temperature to 5° C. over 15 minutes. After 10 minutes stirring, the product was collected by filtration. The filter cake was washed by displacement with IPE (225 g; IPE temperature was 20° C.) and then dried by suction. The product was dried in vacuo at 55° C. to give the title compound as a white solid (100.2 g, 87% over two steps).

### Alternative 6

### (a) [2-(9-Oxa-3,7-diazabicyclo[3.3.1]non-3-yl)ethyl]carbamic acid tert-butyl ester, 2,4,6-trimethylbenzenesulfonic acid salt

[2-(7-Benzyl-9-oxa-3,7-diazabicyclo[3.3.1]non-3-yl)ethyl]carbamic acid tert-butyl ester, 2,4,6-trimethylbenzenesulfonic acid salt (1.00 equiv; 267.56 mmoles; 150.30 g of material that was 3.21% w/w water; see, for example, WO 2004/035592) was added to a hydrogenation vessel. Premixed isopropanol (3.00 moles; 229.30 mL; 180.00 g) and water (4.16 moles; 75.00 mL; 75.00 g) were added, followed by 5% palladium on carbon (4.50 g; ca. 57% w/w water; Engelhard 5398). The vessel was purged with nitrogen (3×) and hydrogen (2×) and then charged to 2 bar hydrogen pressure. Stirring (at 600 rpm) was initiated using a solid stirrer shaft fitted with a retreat curve impeller. Heating of the reaction mixture was started immediately, and the reaction reached the target temperature (65° C.±5° C.) after 15 minutes. After a total reaction time of 50 minutes (including the heat up time) no further hydrogen was taken up (4.846 L had been consumed; theoretical volume consumption: 5.801 L). The reaction was cooled to 25° C. and reaction completion confirmed by thin layer chromatography (1:1 X:DCM as eluent, where X is chloroform:methanol:concentrated aqueous ammonia in the ratios 80:18:2; silica plates, with visualisation by potassium permanganate). (This cooling and sampling step can, if desired, be omitted.) The catalyst was removed by filtration directly into a 500 mL measuring cylinder. The catalyst was then washed with isopropanol (783.75 mmoles; 60.00 mL; 47.10 g).

The total volume of solution in the measuring cylinder was 480 mL, and this was then made up to 500 mL with isopropanol. The weight of solution (containing the title compound) in the measuring cylinder was 461.5 g.

The weight of [2-(7-benzyl-9-oxa-3,7-diazabicyclo[3.3.1]non-3-yl)ethyl]carbamic acid tert-butyl ester, 2,4,6-trimethylbenzenesulfonic acid salt from which that solution was made is 150 g in 500 mL or 30% w/v.

The weight of [2-(7-benzyl-9-oxa-3,7-diazabicyclo[3.3.1]non-3-yl)ethyl]carbamic acid tert-butyl ester, 2,4,6-trimethylbenzenesulfonic acid salt from which that solution was made is 150 g in 461.5 g or 32.5% w/w.

### (b) tent-Butyl 2-{7-[(2S)-3-(4-cyanophenoxy)-2-hydroxypropyl]-9-oxa-3,7-diaza-bicyclo[3.3.1]non-3-yl}ethylcarbamate

A reaction flask was charged with a solution of 3% w/w aqueous sodium carbonate (95.10 mmoles; 326.40 mL; 336.00 g). A portion of the solution of [2-(9-oxa-3,7-diazabicyclo[3.3.1]non-3-yl)-ethyl]-carbamic acid tert-butyl ester, 2,4,6-trimethylbenzenesulfonic acid salt that was generated in part (a) above (350 mL; equates to 105.2 g of the [2-(7-benzyl-9-oxa-3,7-diazabicyclo[3.3.1]non-3-yl)ethyl]carbamic acid tert-butyl ester, 2,4,6-trimethylbenzenesulfonic acid salt precursor; the anhydrous equivalent is 101.8 g) was added. The mixture was heated to 40° C. over the course of 5 minutes, with stirring at 200 rpm. Solid 4-[(2S)-oxiranylmethoxy]benzonitrile (174.67 mmoles; 30.60 g; see, for example WO 01/28992) was added and the reaction heated to 75° C. over 17 minutes. The reaction was held at this temperature for 2 hours. The contents of the flask weighed 678 g. A vacuum was applied, which caused the temperature to fall to ≦50° C., and solvent was removed by distillation. The flask contents now weighed 422 g (thus meaning that 256 g (2.44 rel wt) of solvent had been distilled). Toluene (2.85 moles; 301.88 mL; 263.00 g) was added to the flask contents (which were at 40° C.). An aqueous solution of sodium hydroxide (10% w/w) (315.02 mmoles; 113.63 mL; 126.00 g) was added before the resulting mixture was cooled to 30° C. After 12 minutes, stirring was stopped and the phases allowed to settle (settling occurred within 30 seconds). The phases were separated, at 30° C., leaving interfacial material with the (discarded) aqueous phase. (If desired, a further wash with aqueous base, such as a 10% w/w aqueous solution of sodium hydroxide, can be performed on the organic phase in order to remove traces of mesitylene sulfonic acid.) A solution of 10% w/w aqueous citric acid (163.96 mmoles; 302.83 mL; 315.00 g) was added to the toluene phase. After 7 minutes, stirring was stopped and the phases allowed to settle (settling occurred within 20 seconds). The phases were separated, at 29° C., leaving interfacial material with the (discarded) upper (organic) phase. 4-Methyl-2-pentanol (MIBC) (2.88 moles; 366.58 mL; 294.00 g) was added, followed by an aqueous solution of sodium hydroxide (15% w/w) and sodium chloride (7.5% w/w) (210.00 g). After 2 minutes, stirring was stopped and the phases allowed to settle (settling occurred within 60 seconds). The phases were separated, at 37° C., leaving interfacial material with the (discarded) lower (aqueous) phase. An aqueous solution of sodium chloride (20% w/w) (179.66 mmoles; 45.74 mL; 52.50 g) was added and stirred. After 2 minutes, stirring was stopped and the phases allowed to settle (settling occurred within 80 seconds). The phases were separated, at 35° C., leaving any interfacial material with the (discarded) lower (aqueous) phase. The contents of the flask weighed 395 g. The solution was distilled under vacuum, which led to the collection of 19 mL of water and 58 mL of MIBC. The flask contents now weighed 317 g (thus meaning that therefore 78 g (0.75 rel wt) had been removed by distillation). The remaining solution was filtered into a clean vessel and rinsed through with MIBC (411.05 mmoles; 52.37 mL; 42.00 g). The contents of the new flask weighed 351 g. The solution was left overnight (for convenience). During this time some crystallisation occurred. The mixture was heated to 60° C. and all material dissolved. The solution was distilled under vacuum at ≦70° C., leading to the collection of 183 mL of liquid (based on a density of MIBC of 0.802 this is 1.4 rel wt). Diisopropyl ether (DIPE) (3.24 moles; 457.00 mL; 331.32 g) was added to the hot (70° C.) MIBC solution, which caused the temperature of the mixture to fall to 52° C. The solution was re-heated to 60° C. and then allowed to cool naturally. After 27 minutes, the flask contents had reached 45° C. and seed crystals (56 mg) were added. The mixture was allowed to cool to 27° C. (this took 2 hours) by which time a large amount of precipitate was present. The mixture was cooled to 5° C. over the course of 24 minutes and then held at this temperature for 1 hour. The product was then collected by filtration (a process that took 45 seconds) and was washed with cold (5° C.) DIPE (1.54 moles; 217.24 mL; 157.50 g), which took 30 seconds. The filter cake was pulled as dry as possible on the filter (10 minutes). The damp material (99 g) was then dried in vacuo (at 55° C.) to constant weight (which took 1.5 hours). This gave the title compound as a white solid (68.3 g, 84%).

### Example 2

### tert-Butyl (2-{7-[2-(4-cyano-2-fluorophenoxy)ethyl]-9-oxa-3,7-diazabicyclo-[3.3.1]non-3-yl}ethyl)carbamate

### Alternative 1

To [2-(7-benzyl-9-oxa-3,7-diazabicyclo[3.3.1]non-3-yl)ethyl]carbamic acid tert-butyl ester, 2,4,6-trimethylbenzenesulfonic acid salt (100 g; see, for example, WO 2004/035592), was added isopropanol (300 mL) and water (100 mL). To this was added 5% w/w palladium on carbon (4 g) (approximately 60% wet paste). This was heated to 65° C. and was hydrogenated at 3.5 bar. The reaction mixture was held at 65° C. for approximately sixteen hours before being cooled to 20° C.; total volume of hydrogen uptake was 4 L. The catalyst was removed by filtration and the catalyst was washed with isopropanol (50 mL). The combined organic filtrate and isopropanol catalyst washing were concentrated in vacuo. This gave a white crystalline solid, which was taken up in acetonitrile (1.28 L). To this was added 4-(2-bromoethoxy)-3-fluorobenzonitrile (43.5 g; see Preparation A above) and potassium carbonate (250 g). The reaction was heated to reflux (approximately 80° C.), and held at this temperature for four hours. The reaction mixture was cooled to approximately 20° C. The reaction mixture was filtered, and the filter cake was washed with acetonitrile (250 mL). The combined organic filtrate and acetonitrile washings were concentrated in vacuo, and the residue was taken up in toluene (345 mL). This was then heated to 30° C. and kept at this temperature until the end of the extractive work-up. To the toluene solution was added a solution of sodium hydroxide (12 g) dissolved in water (110 mL). The layers were separated, and the lower (aqueous) phase was discarded. To the retained organic layer was added a solution of citric acid (30 g) dissolved in water (270 mL). The layers were separated, and the upper (organic) layer was discarded. To the retained aqueous layer was added ethyl acetate (330 mL), and a solution of sodium hydroxide (60 g) and sodium chloride (30 g) dissolved in water (310 mL). The layers were separated, and the lower (aqueous) phase was discarded. To the retained organic layer was added a solution of sodium chloride (10 g) dissolved in water (40 mL). The layers were separated, and the lower (aqueous) phase was discarded. The ethyl acetate layer was dried over magnesium sulfate (10 g), filtered and the drying agent was washed with ethyl acetate (220 mL). The combined organic filtrate and ethyl acetate washings were concentrated in vacuo, to yield the title compound as a light yellow oil containing white crystalline parts within it (72.00 g, 93% yield).

Crystallisation of the title compound can be carried out, if necessary, using the following method.

To tert-butyl (2-{7-[2-(4-cyano-2-fluorophenoxy)ethyl]-9-oxa-3,7-diazabicyclo-[3.3.1]non-3-yl}ethyl)carbamate (77 g) was added diisopropylether (385 mL) and isopropanol (77 mL). This mixture was heated to 65° C., and held at this temperature for fifteen minutes before being cooled to 5° C. over ninety minutes. Crystallisation was noticed at between 15 and 10° C. The mixture was held at 5° C. for two hours before being filtered and washed with cold diisopropylether (80 mL, 5° C.). The damp solid was dried in vacuo, at 35° C., for approximately nineteen hours, to give the crystallised title compound as an off-white solid (54.5 g, 71% yield). ¹H-NMR (CDCl₃, 300 MHz) δ 7.48-7.30 (m), 7.15-6.96 (m), 6.30-6.01 (m), 4.58-4.23 (m), 3.91-3.82 (m), 3.27-3.08 (m), 3.04-2.87 (m), 2.85-2.59 (m), 2.48-2.35 (m), 1.40 (s).

### Alternative 2

### Sample 1

To [2-(7-benzyl-9-oxa-3,7-diazabicyclo[3.3.1]non-3-yl)ethyl]carbamic acid tert-butyl ester, 2,4,6-trimethylbenzenesulfonic acid salt (148 g; see, for example, WO 2004/035592), was added isopropanol (450 mL) and water (150 mL). To this was added 5% w/w palladium on carbon (7.5 g, approximately 60% wet paste).

The resulting mixture was heated to 65° C. and was hydrogenated at 3.5 bar. The reaction mixture was maintained at 65° C., for approximately fourteen hours before being cooled to 20° C.; total volume of hydrogen uptake was 5.9 L. The catalyst was removed by filtration, and the catalyst was washed with isopropanol (75 mL). The combined organic filtrate and isopropanol catalyst washings were concentrated in vacuo and the resulting residue (white crystalline solid) was taken up in acetonitrile (1.9 L). To this was added 2-(4-cyano-2-fluorophenoxy)ethyl toluene-4-sulfonate (88.3 g; see Preparation B above) and potassium carbonate (91 g). The reaction mixture was heated to reflux (approximately 80° C.), and was maintained at this temperature for eight hours, before cooling to room temperature (approximately 20° C.). The reaction mixture was filtered and the filter cake was washed with acetonitrile (190 mL). The combined filtrate and acetonitrile cake washings were concentrated in vacuo and the resulting residue was taken up in toluene (850 mL). To this was added a solution of sodium hydroxide (26.6 g) dissolved in water (240 mL). The layers were separated, and the lower (aqueous) layer was discarded. To the retained organic layer was added a solution of citric acid (44.4 g) dissolved in water (400 mL). The layers were separated, and the upper (organic layer) was discarded. To the retained aqueous layer was added ethyl acetate (1.25 L) and a solution of sodium hydroxide (89 g) and sodium chloride (44.4 g) dissolved in water (460 mL). The layers were separated, and the lower (aqueous) layer was discarded. To the retained organic layer was added a solution of sodium chloride (15 g) dissolved in water (60 mL). The layers were separated, and the lower (aqueous) layer was discarded. The retained organic layer was dried over magnesium sulfate (75 g). The magnesium sulfate was removed by filtration and the drying agent was washed with ethyl acetate (410 mL). The combined organic filtrate and ethyl acetate washing were concentrated in vacuo to yield a yellow oil (97 g). This oil was taken up in diisopropylether (485 mL) and isopropanol (100 mL). This was then heated to reflux (approximately 68° C.). At reflux all of the material had dissolved, and the reaction mixture was cooled to room temperature (approximately 20° C.). The mixture was further cooled to 5° C. before being filtered and washed with cold diisopropylether (200 mL, 5° C.). The damp solid was dried in vacuo, at 35° C., to give the title compound as an off white solid (60 g, 52.4% yield).

### Sample 2

To [2-(7-benzyl-9-oxa-3,7-diazabicyclo[3.3.1]non-3-yl)ethyl]carbamic acid tert-butyl ester, 2,4,6-trimethylbenzenesulfonic acid salt (173 g; see, for example, WO 2004/035592), was added isopropanol (530 mL) and water (175 mL). Then 5% w/w palladium on carbon (8.7 g of approximately 60% wet paste) was added. The reaction mixture was heated to 65° C. and was hydrogenated at 3.5 bar. The reaction mixture was maintained at 65° C. for two hours, and was then cooled to 20° C.; total volume of hydrogen uptake was 7.1 L. The catalyst was removed by filtration, and the catalyst was washed with isopropanol (90 mL). The combined organic filtrate and isopropanol catalyst washings were concentrated in vacuo, and the residue (white crystalline solid) was taken up in acetonitrile (2.2 L). To this was added 2-(4-cyano-2-fluorophenoxy)ethyl toluene-4-sulfonate (103.3 g; see Preparation B above) and potassium carbonate (106.5 g). This was then heated to reflux (approximately 80° C.), over approximately half an hour. The reaction mixture was maintained at 80° C. for eight hours, before cooling to room temperature (approximately 20° C.). The reaction mixture was filtered and the filter cake was washed with acetonitrile (220 mL). The combined filtrate and acetonitrile cake washings were concentrated in vacuo and the resulting residue was taken up in toluene (1 L). To this was added a solution of sodium hydroxide (31.2 g) dissolved in water (280 mL). The layers were separated, and the lower (aqueous) layer was discarded. To the retained organic layer was added a solution of citric acid (52 g) dissolved in water (470 mL). The layers were separated, and the upper (organic) layer was discarded. To the retained aqueous layer was added ethyl acetate (1.45 L) and a solution of sodium hydroxide (104 g) and sodium chloride (52 g) dissolved in water (540 mL). The layers were separated, and the lower (aqueous) layer was discarded. To the retained organic layer was added a solution of sodium chloride (17.3 g) dissolved in water (70 mL). The layers were separated, and the lower (aqueous) layer was discarded. The organic layer was dried over magnesium sulfate (87 g), filtered and the drying agent washed with ethyl acetate (480 mL). The combined organic filtrate and ethyl acetate cake washings were concentrated in vacuo to yield a yellow oil (113 g). To this oil was added diisopropylether (600 mL) and isopropanol (110 mL), this mixture was heated to reflux (approximately 68° C.). At reflux, all of the material had dissolved, and the reaction mixture was cooled to room temperature (approximately 20° C.). The reaction mixture was further cooled to 5° C., and the reaction mixture was filtered. The solid was washed with cold diisopropylether (240 mL, 5° C.). The damp solid was dried in a vacuum oven, at 35° C., to give the title compound as an off white solid (79 g, 59% yield).

### Sample 3

The final filtrates and the washes from the above two procedures, were combined and concentrated in vacuo to give a further 80 g of the crude title compound. To this crude mixture was added diisopropylether (530 mL) and isopropanol (70 mL), which gave a mixture that was then heated to 65° C. At 65° C., all of the material had dissolved and the mixture was cooled to room temperature (approximately 20° C.). The mixture was then further cooled to 5° C. before it was filtered and the solid was washed with cold diisopropylether (70 mL, 5° C.). The damp solid was dried in vacuo, at 35° C., to give purified title compound as an off white solid (25 g, 31.3% yield).

The title compound samples resulting from the above three procedures (i.e. Samples 1 to 3: 60 g, 79 g and 25 g, respectively) were combined. To the combined mixture was added diisopropylether (820 mL) and isopropanol (82 mL). This mixture was heated to 65° C., at which temperature a solution had formed. The reaction mixture was cooled over three hours to room temperature (approximately 20° C.). Crystallisation was noticed at between 45 and 40° C. The mixture was further cooled to 5° C. over twenty minutes and held at 5° C. for twenty more minutes. The reaction mixture was filtered and the solid was washed with cold diisopropylether (165 mL, 5° C.). The damp solid was dried in vacuo, at 35° C., to give recrystallised title compound as an off white solid (149.3 g, 91% yield).

¹H-NMR (CDCl₃, 300 MHz) δ H 7.49-7.29 (m, 2H), 7.16-6.94 (m, 1H), 6.31-6.02 (m, 1H), 4.57-4.21 (m, 2H), 3.93-3.82 (m, 2H), 3.28-3.07 (m, 2H), 3.05-2.87 (m, 2H), 2.85-2.62 (m, 8H), 2.49-2.37 (m, 2H), 1.43 (s, 91-1).

### Alternative 3

To [2-(7-benzyl-9-oxa-3,7-diazabicyclo[3.3.1]non-3-yl)ethyl]carbamic acid tert-butyl ester, 2,4,6-trimethylbenzenesulfonic acid salt (60 g; see, for example, WO 2004/035592) was added a solution of isopropanol (92 mL) and water (30 mL). To this was added 5% w/w palladium on carbon (2.4 g of 61% wet paste). The reaction mixture was heated to 65° C. and was hydrogenated at 2.5 bar. The reaction mixture was held at 65° C. for twenty minutes, and was then cooled to 20° C.; total volume of hydrogen uptake was 2.2 L. The catalyst was removed by filtration and the catalyst was washed with isopropanol (31 mL). The organic filtrate and the isopropanol catalyst washings were combined. To this was added 2-(4-cyano-2-fluorophenoxy)ethyl toluene-4-sulfonate (35.1 g; see Preparation B above), and a solution of sodium carbonate (63 g) dissolved in water (186 mL). The reaction mixture was heated to 75° C., at approximately 1° C. per minute. The reaction mixture was held at 75° C. for twelve hours then cooled to 20° C., at approximately 1° C. per minute. The reaction mixture was reduced in volume by reduced pressure distillation (at less than 50° C.), and approximately 150 mL of solvent was removed. To the remaining reaction mixture was added toluene (175 mL) and the reaction temperature was adjusted to 30° C. and kept at this temperature until the end of the extractive work up. To the toluene solution was added a solution of sodium hydroxide (10.8 g) dissolved in water (98 mL). The layers were separated and the lower (aqueous) layer was discarded. To the retained organic layer was added a solution of citric acid (18.0 g) dissolved in water (162 mL). The layers were separated and the upper (organic) layer was discarded. To the retained aqueous layer was added 4-methylpentan-2-ol (210 mL), and a solution of sodium hydroxide (36 g) and sodium chloride (18 g) dissolved in water (186 mL). The layers were separated and the lower (aqueous) layer was discarded. To the retained organic layer was added a solution of sodium chloride (6 g) dissolved in water (24 mL). The layers were separated and the lower (aqueous) layer was discarded. The resulting mixture was reduced in volume by reduced pressure distillation (at less than 70° C., resulting in the removal of approximately 55 mL of solvent). This was then filtered to a clean vessel and was washed with 4-methyl-pentan-2-ol (30 mL). The mixture was reduced in volume by reduced pressure distillation (at less than 70° C.), and approximately 155 mL of solvent was removed. To the residue was added diisopropylether (560 mL), whilst maintaining the temperature above 55° C. The mixture was cooled to 20° C., at approximately 0.25° C. per minute, then held at 20° C. for approximately sixteen hours. The mixture was cooled to 5° C., at approximately 0.25° C. per minute, and was held at 5° C. for approximately an hour. The mixture was filtered and the product was washed with cold diisopropylether (125 mL, 5° C.). The damp solid was dried in vacuo, at 35° C., for approximately twenty-two hours, to give the title compound as a white, crystalline solid (29 g, 63% yield).

Recrystallisation of the title compound can be carried out, if necessary, using the following method.

To tert-butyl (2-{7-[2-(4-cyano-2-fluorophenoxy)ethyl]-9-oxa-3,7-diazabicyclo-[3.3.1]non-3-yl}ethyl)carbamate (164 g) was added diisopropylether (820 mL) and isopropanol (82 mL). This mixture was then heated to 65° C., at which point a solution had formed. The reaction mixture was cooled to room temperature (approximately 20° C.). Crystallisation was noticed at between 45 and 40° C. The mixture was further cooled to 5° C. before it was filtered and the solid was washed with cold diisopropylether (165 mL, 5° C.). The damp solid was dried in vacuo, at 35° C., for approximately eighteen hours, to give recrystallised title compound (149.3 g; 91%).

¹H NMR (400 MHz, CD₃OD): δ 7.53 (d, J=9.8 Hz, 2H), 7.29 (t, J=8.2 Hz, 1H), 4.38 (t, J=5.9 Hz, 2H), 3.89-3.82 (m, 2H), 3.17 (t, J=6.3 Hz, 2H), 3.01 (d, J=11.5 Hz, 2H), 2.86 (d, J=11.3 Hz, 2H), 2.78 (t, J=6.0 Hz, 2H), 2.67-2.60 (m, 2H), 2.60-2.53 (m, 2H), 2.39 (t, J=6.2 Hz, 2H), 1.41 (s, 9H).

### Alternative 4

To [2-(7-benzyl-9-oxa-3,7-diazabicyclo[3.3.1]non-3-yl)ethyl]carbamic acid tert-butyl ester, 2,4,6-trimethylbenzenesulfonic acid salt (60 g; see, for example, WO 2004/035592) was added a solution of isopropanol (90 mL) and water (30 mL). To this was added 5% w/w palladium on carbon (2.4 g of 61% wet paste). The reaction mixture was heated to 65° C. and was hydrogenated at 2.5 bar. The reaction mixture was maintained at 65° C. for approximately forty-five minutes and was then cooled to 20° C.; total volume of hydrogen uptake was 2.2 L. The catalyst was removed by filtration and the catalyst was washed with isopropanol (31 mL). To the combined organic filtrate and the isopropanol catalyst washings was added 2-(4-cyano-2-fluorophenoxy)ethyl toluene-4-sulfonate (35.1 g; see Preparation B above) and a solution of sodium carbonate (63 g) dissolved in water (186 mL). The reaction mixture was heated to 75° C., at approximately 1° C. per minute, then held at this temperature for twelve hours, before being cooled to 20° C. (at approximately 1° C. per minute). The reaction mixture was reduced in volume by reduced pressure distillation (at less than 50° C.), and approximately 140 mL of solvent was removed. To the remaining mixture was added toluene (172 mL), and the reaction temperature was adjusted to 30° C. and kept at this temperature until the end of the extractive work up. To the toluene solution was added a solution of sodium hydroxide (10.8 g) dissolved in water (97 mL). The layers were separated and the lower (aqueous) layer was discarded. This extraction with aqueous sodium hydroxide was repeated once more, the lower (aqueous) layer again being discarded. To the retained organic layer was added a solution of citric acid (18 g) dissolved in water (162 mL). The layers were separated and the upper (organic) layer was discarded. To the retained aqueous layer was added ethyl acetate (210 mL) and a solution of sodium hydroxide (36 g) and sodium chloride (18 g) dissolved in water (186 mL). The layers were separated and the lower aqueous layer was discarded. To the retained organic layer was added a solution of sodium chloride (6 g) dissolved in water (24 mL). The layers were separated and the lower aqueous layer was discarded. The retained organic layer was dried over magnesium sulfate (30 g). The inorganic solids were removed by filtration, and washed with ethyl acetate (30 mL). The combined filtrate and washings were concentrated in vacuo, at less than 50° C., to give a colourless oil (40 g). To this oil was added diisopropylether (175 mL) and isopropanol (35 mL). This mixture was heated to 65° C., at approximately 1° C. per minute. The temperature was then maintained at 65° C. for fifteen minutes. The mixture was then cooled to 20° C. (at approximately 0.25° C. per minute), held at 20° C. for approximately sixteen hours, then cooled to 5° C. (at approximately 0.25° C. per minute), before being held at this final temperature for approximately one hour. The reaction mixture was filtered and the solid was washed with cold diisopropylether (36 mL, 5° C.). The damp solid was dried in vacuo, at 35° C., to give the title compound as a white solid (28.2 g, 61% yield).

### Alternative 5

To [2-(7-benzyl-9-oxa-3,7-diazabicyclo[3.3.1]non-3-yl)ethyl]carbamic acid tert-butyl ester, 2,4,6-trimethylbenzenesulfonic acid salt (300 g; see, for example, WO 2004/035592) was added isopropanol (460 mL) and water (150 mL). To the resulting mixture was added 5% w/w palladium on carbon (12 g of approximately 60% wet paste). The mixture was then heated to 65° C. and was hydrogenated at 2.5 bar. The reaction mixture was held at 65° C. for approximately twenty minutes before being cooled to 20° C.; total volume of hydrogen uptake was 11.4 L. The catalyst was removed by filtration and was washed with isopropanol (150 mL). The organic filtrate and the isopropanol catalyst washings were combined. To this was added 2-(4-cyano-2-fluorophenoxy)ethyl toluene-4-sulfonate (175.5 g; see Preparation B above) and a solution of sodium carbonate (315 g) dissolved in water (930 mL). The reaction mixture was heated to 75° C., at which temperature it was held for twelve hours before being cooled to 20° C. The reaction mixture was reduced in volume by reduced pressure distillation (at less than 50° C.), and approximately 650 mL of solvent was removed. To the remaining reaction mixture was added toluene (860 mL) and the reaction temperature was adjusted to 30° C. and kept at this temperature until the end of the extractive work-up. To the toluene solution was added a solution of sodium hydroxide (54 g) dissolved in water (485 mL). The layers were separated and the lower (aqueous) layer was discarded. To the retained (organic) solution was added a solution of sodium hydroxide (54 g) dissolved in water (485 mL). The layers were separated and the lower (aqueous) layer was discarded. To the retained (organic) layer was added a solution of citric acid (90 g) dissolved in water (810 mL). The layers were separated and the upper (organic) layer was discarded. To the retained (aqueous) layer was added ethyl acetate (1.05 L), and a solution of sodium hydroxide (180 g) and sodium chloride (90 g) dissolved in water (930 mL). The layers were separated and the lower (aqueous) layer was discarded. To the retained (organic) layer was added a solution of sodium chloride (30 g) dissolved in water (120 mL). The layers were separated and the lower (aqueous) layer was discarded. The retained (organic) layer was dried over magnesium sulfate (150 g). The inorganic solids were removed by filtration, and washed with ethyl acetate (150 mL). The combined filtrate and washings were concentrated in vacuo, at less than 50° C., to give crude title compound as a yellow solid (201 g).

The above procedure was repeated three further times (starting once with 300 g of [2-(7-benzyl-9-oxa-3,7-diazabicyclo[3.3.1]non-3-yl)ethyl]carbamic acid tert-butyl ester 2,4,6-trimethylbenzenesulfonate and twice with 450 g of this material) to provide batches of 200, 304 and 300 g of crude title compound.

The four batches of crude title compound mentioned above (0.2 kg, 0.2 kg, 0.3 kg and 0.3 kg) were combined. Diisopropylether (5 L) and isopropanol (1 L) were added to the combined material. The resulting mixture was heated to 65° C., at which temperature a solution had formed. The reaction mixture was cooled over approximately six hours to room temperature (approximately 20° C.). Crystallisation was noticed at approximately 37° C. For convenience, the reaction mixture was held at 20° C. for approximately sixteen hours. The reaction mixture was further cooled to 5° C. over fifty minutes and held at 5° C. for five minutes. The reaction mixture was filtered and the solid was washed with cold diisopropylether (1 L, 5° C.). The damp solid was dried in vacuo, at 35° C., to give purified title compound as an off-white solid (741 g, 74% yield).

¹H NMR (400 MHz, CD₃OD): δ 7.53 (d, J=9.5 Hz, 2H), 7.30 (d, J=8.2 Hz, 1H), 4.38 (t, J=5.9 Hz, 2H), 3.88-3.82 (m, 2H), 3.17 (t, J=6.2 Hz, 1H), 3.01 (d, J=11.2 Hz, 2H), 2.86 (d, J=12.1 Hz, 2H), 2.78 (t, J=5.8 Hz, 2H), 2.67-2.60 (m, 2H), 2.60-2.54 (m, 2H), 2.39 (t, J=6.2 Hz, 2H), 1.37 (s, 9H).

### Alternative 6

To [2-(7-benzyl-9-oxa-3,7-diazabicyclo[3.3.1]non-3-yl)ethyl]carbamic acid tert-butyl ester 2,4,6-trimethylbenzenesulfonate (30 g; see WO 2004/035592) was added a solution of isopropanol (46 mL) and water (15 mL). To this was added 5% w/w palladium on carbon (1.2 g of 61% wet paste). The reaction mixture was heated to 65° C. and was hydrogenated at 2.5 bar. The reaction mixture was held at 65° C. for twenty minutes, and was then cooled to 20° C.; total volume of hydrogen uptake was 1.1 L. The catalyst was removed by filtration and was then washed with isopropanol (15 mL). The organic filtrate and the isopropanol catalyst washings were combined. To this was added 2-(4-cyano-2-fluorophenoxy)ethyl toluene-4-sulfonate (17.55 g; see Preparation B above), and a solution of sodium carbonate (5.94 g) dissolved in water (93 mL). The reaction mixture was heated to 75° C., at approximately 1° C. per minute. The reaction mixture was held at 75° C. for twelve hours then cooled to 20° C., at approximately 1° C. per minute. The reaction mixture was reduced in volume by reduced pressure distillation (at less than 50° C.), and approximately 60 mL of solvent was removed. To the remaining reaction mixture was added toluene (75 mL) and the reaction temperature was adjusted to 30° C. and kept at this temperature until the end of the extractive work up. To the toluene solution was added a solution of sodium hydroxide (3.6 g) dissolved in water (32 mL). The layers were separated and the lower (aqueous) layer was discarded. To the retained organic layer was added a solution of citric acid (9 g) dissolved in water (81 mL). The layers were separated and the upper (organic) layer was discarded. To the retained aqueous layer was added 4-methyl-pentan-2-ol (104 mL), and a solution of sodium hydroxide (18 g) and sodium chloride (9 g) dissolved in water (93 mL). The layers were separated and the lower (aqueous) layer was discarded. To the retained organic layer was added a solution of sodium chloride (3 g) dissolved in water (12 mL). The layers were separated and the lower (aqueous) layer was discarded. The resulting mixture was reduced in volume by reduced pressure distillation (at less than 70° C., resulting in the removal of approximately 15 mL of solvent). This was then filtered to a clean vessel and was washed with 4-methylpentan-2-ol (15 mL). The mixture was reduced in volume by reduced pressure distillation (at less than 70° C.), and approximately 90 mL of solvent was removed. To the residue was added diisopropylether (280 mL), whilst maintaining the temperature above 40° C. The mixture was re-heated to 55° C. before being cooled to 20° C. (at approximately 0.25° C. per minute), at which temperature it was held for approximately fourteen hours. The mixture was then cooled to 5° C., at approximately 0.25° C. per minute, and was held at 5° C. for approximately two hours. The mixture was filtered and the filter cake was washed with cold diisopropylether (62 mL, 5° C.). The damp solid was dried in vacuo (at 35° C. for approximately twenty-two hours) to give the title compound as a white, crystalline solid (17.8 g, 77% yield).

### Alternative 7

To [2-(7-benzyl-9-oxa-3,7-diazabicyclo[3.3.1]non-3-yl)ethyl]carbamic acid tert-butyl ester, 2,4,6-trimethylbenzenesulfonic acid salt (101 g; see WO 2004/035592) was added a solution of isopropanol (152 mL) and water (50 mL). To this was added 5% w/w palladium on carbon (4 g of 61% wet paste). The reaction mixture was heated to 65° C. and was hydrogenated at 2.5 bar. The reaction mixture was held at 65° C. for approximately one hour, and was then cooled to 20° C.; total volume of hydrogen uptake was 3.8 L. The catalyst was removed by filtration and washed with isopropanol (50 mL). The organic filtrate and the isopropanol catalyst washings were combined. To this was added 2-(4-cyano-2-fluorophenoxy)ethyl toluene-4-sulfonate (58.95 g; see Preparation B above), and a solution of sodium carbonate (20.01 g) dissolved in water (310 mL). The reaction mixture was heated to 75° C. The reaction mixture was held at 75° C. for twelve hours then cooled to 20° C. The reaction mixture was reduced in volume by reduced pressure distillation (at less than 45° C.), and approximately 210 mL of solvent was removed. To the remaining reaction mixture was added toluene (290 mL) and the reaction temperature was adjusted to 30° C. and kept at this temperature until the end of the extractive work up. To the toluene solution was added a solution of sodium hydroxide (18.01 g) dissolved in water (162 mL). The layers were separated and the lower (aqueous) layer was discarded. To the retained organic layer was added a solution of sodium hydroxide (18.18 g) dissolved in water (162 mL). The layers were separated and the lower (aqueous) layer was discarded. To the retained organic layer was added a solution of citric acid (30.15 g) dissolved in water (270 mL). The layers were separated and the upper (organic) layer was discarded. To the retained aqueous layer was added 4-methylpentan-2-ol (350 mL), and a solution of sodium hydroxide (60.32 g) and sodium chloride (30.27 g) dissolved in water (310 mL). The layers were separated and the lower (aqueous) layer was discarded. To the retained organic layer was added a solution of sodium chloride (10.07 g) dissolved in water (40 mL). The layers were separated and the lower (aqueous) layer was discarded. The resulting mixture was reduced in volume by reduced pressure distillation (at less than 60° C., resulting in the removal of approximately 180 mL of solvent), filtered to a clean vessel and then washed with 4-methyl-pentan-2-ol (50 mL). The mixture was reduced in volume by reduced pressure distillation (at less than 60° C.), and approximately 124 mL of solvent was removed. To the residue was added diisopropylether (935 mL), whilst maintaining the temperature above 55° C. The mixture was cooled to 20° C. and then to 5° C., at which temperature it was held for approximately an hour. The mixture was filtered and the product was washed with cold diisopropylether (200 mL, 5° C.). The damp solid was dried in vacuo, at 35° C., for approximately twenty-five hours, to give the title compound as a white, crystalline solid (51.4 g, 66% yield).

¹H NMR (400 MHz, CD₃OD): δ 7.50 (d, J=9.5 Hz, 2H), 7.27 (t, J=8.3 Hz, 1H), 4.36 (t, J=5.8 Hz, 2H), 3.83 (t, J=3.5 Hz, 2H), 3.15 (t, J=6.2 Hz, 2H), 2.99 (d, J=11.5 Hz, 2H), 2.84 (d, J=11.3 Hz, 2H), 2.76 (t, J=6.0 Hz, 2H), 2.66-2.50 (m, 4H), 2.37 (t, J=6.3 Hz, 2H), 1.36 (s, 9H).

### Example 3

### tert-Butyl (3-{7-[3-(4-cyanophenoxy)propyl]-9-oxa-3,7-diazabicyclo[3.3.1]non-3-yl}propyl)carbamate, L-tartaric acid salt

To [3-(7-Benzyl-9-oxa-3,7-diazabicyclo[3.3.1]non-3-yl)propyl]carbamic acid tert-butyl ester, 4-chlorobenzenesulfonic acid salt (50.20 g, 88.36 mmol; see Preparation C above) was added a mixture of propan-2-ol (150 mL) and water (50 mL), followed by 5% palladium on carbon (2.53 g, 5% rel. wt., ca. 60% water wet paste). The resulting mixture was hydrogenated at 2.5 bar and immediately heated to 50° C. Once the reaction had taken up the required amount of hydrogen, it was cooled to room temperature. TLC (1:1 dichloromethane:Solvent X, potassium permanganate stain, X=80:18:2 chloroform:methanol: 35% aqueous ammonia) showed that the reaction was complete. The catalyst was removed by filtration and washed with propan-2-ol (75 mL). To the resulting filtrate and catalyst washing was added 1 M aqueous sodium carbonate (115 mL). The resulting mixture was heated to 55° C. and 3-(4-cyanophenoxy)propyl 4-toluene-sulfonate (30.71 g, 92.67 mmol; see Preparation D above) was added. This provided a mixture that was then heated at reflux for 4 hours. TLC (1:1 dichloromethane:X) showed the reaction was complete. Solvent (236 mL) was removed by distillation under reduced pressure, keeping the temperature below 50° C. Toluene (220 mL) and 1 M sodium hydroxide (100 mL) were added and the resulting mixture was cooled to room temperature before further toluene (30 mL) and 1 M sodium hydroxide (50 mL) were added. The phases were separated and 10% w/w citric acid (250 mL) was added to the retained organic. After being stirred together at room temperature for 15 minutes, the phases were again separated. To the retained aqueous phase was added isopropyl acetate (550 mL) and 5 M sodium hydroxide (150 mL). Stirring at room temperature was then undertaken for 10 minutes. The phases were separated once more and the organic phase retained and washed with 20% w/w sodium chloride solution (50 mL). Solvent (100 mL) was removed from the organic phase by distillation under reduced pressure, keeping the temperature below 50° C. The remaining mixture was filtered whilst hot to remove insoluble material, which was then washed through with isopropyl acetate (50 mL). (At this stage, if desired, tert-butyl (3-{7-[3-(4-cyanophenoxy)propyl]-9-oxa-3,7-diazabicyclo[3.3.1]non-3-yl}-propyl)carbamate in neutral form can be isolated by concentration of the resulting filtrate.) The solution was re-heated to 50° C. before L-tartaric acid (13.42 g, 88.52 mmol), dissolved (by warming) in ethanol (150 mL), was added over the course of 30 minutes. The resulting mixture was cooled to room temperature, causing crystallisation of the product from solution. The solution was cooled to 5° C., the product collected by filtration and the filter cake washed with isopropyl acetate (150 mL). The product was dried as far as possible on the filter, then oven dried in vacuo (40° C., 24 h) to give the sub-title compound as a white solid (44.00 g, 73.99 mmol, 84%).

¹H NMR (400 MHz, CD₃OD) δ 7.65 (dd, J=6.9, 1.8 Hz, 2H), 7.06-7.02 (m, 2H), 4.38 (s, 2H), 4.19 (s, 2H), 4.14 (t, J=6.0 Hz, 2H), 3.51 (d, J=12.3 Hz, 2H), 3.27 (s, 1H), 3.11 (t, J=5.9 Hz, 4H), 3.02 (t, J=7.9 Hz, 2H), 2.90 (d, J=11.8 Hz, 2H), 2.64 (t, J=6.2 Hz, 2H), 2.24 (dd, J=10.1, 5.8 Hz, 2H), 1.74 (t, J=6.0 Hz, 2H), 1.45 (s, 9H).

¹H NMR (400 MHz, D₂O) δ 7.75 (d, J=9.0 Hz, 2H), 7.11 (d, J=8.7 Hz, 2H), 4.53 (s, 2H), 4.30 (s, 2H), 4.24 (t, J=5.6 Hz, 3H), 3.57 (d, J=12.6 Hz, 2H), 3.40 (d, J=12.3 Hz, 2H), 3.12 (d, J=12.3 Hz, 2H), 3.01 (t, J=7.2 Hz, 4H), 2.94 (t, J=6.2 Hz, 3H), 2.63 (t, J=7.2 Hz, 2H), 2.21 (quintet, J=6.5 Hz, 2H), 1.66 (q, J=6.7 Hz, 2H), 1.43 (d, J=10.8 Hz, 9H).

### Abbreviations

Et=ethyl
eq.=equivalents
h=hour(s)
IPA=iso-propyl alcohol
IPE=diisopropyl ether
Me=methyl
MIBC=4-methyl-2-pentanol
MIBK=methyl isobutyl ketone
min.=minute(s)

### MPa=Mega Pascal

Pd/C=palladium on carbon
Pt/C=platinum on carbon
TLC=thin layer chromatography

Prefixes n-, s-, t- and tert- have their usual meanings: normal, secondary, iso, and tertiary.

1. A process for the preparation of a sulfonic acid salt of formula I, 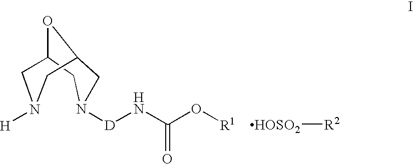
or a solvate thereof;
wherein
R¹ represents C₁₋₆ alkyl (optionally substituted by one or more substituents selected from —OH, halo, cyano, nitro and aryl) or aryl;
D represents optionally branched C₂₋₆ alkylene, provided that it does not represent 1,1-C₂₋₆ alkylene;
R² represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy; and
wherein each aryl group, unless otherwise specified, is optionally substituted;
which process comprises hydrogenating a sulfonic acid salt of formula II, 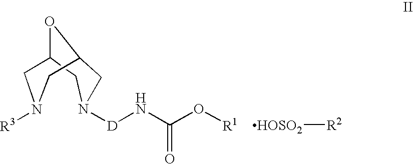
or a solvate thereof;
wherein R³ represents an amino protective group that is labile to hydrogenation, and R¹, R² and D are as defined above,
in the presence of a solvent system consisting essentially of water, a C₃₋₅ secondary alkyl alcohol and no more than 20% v/v of another organic solvent.

2. A process as claimed in claim 1, wherein the process is performed so as to provide a solution of a salt of formula I in a solvent system consisting essentially of water, a C₃₋₅ secondary alkyl alcohol and no more than 15% v/v of another organic solvent.

3. A process for the preparation of a compound of formula IX, 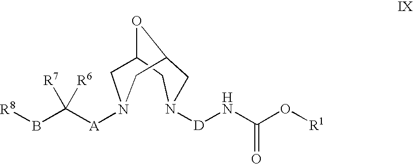
or a pharmaceutically-acceptable derivative thereof;
wherein
R¹ represents C₁₋₆ alkyl (optionally substituted by one or more substituents selected from —OH, halo, cyano, nitro and aryl) or aryl;
D represents optionally branched C₂₋₆ alkylene, provided that it does not represent 1,1-C₂₋₆ alkylene;
R⁶ represents H, halo, C₁₋₆ alkyl, —OR⁹, -E-N(R¹⁰)R¹¹ or, together with R⁷, represents ═O;
R⁷ represents H, C₁₋₆ alkyl or, together with R⁶, represents ═O;
R⁹ represents H, C₁₋₆ alkyl, -E-aryl, -E-Het¹, —C(O)R^{12a}, —C(O)OR^{12b} or —C(O)N(R^{13a})R^{13b};
R¹⁰ represents H, C₁₋₆ alkyl, -E-aryl, -E-Het¹, —C(O)R^{12a}, —C(O)OR^{12b}, —S(O)₂R^{12c}, —[C(O)]ₚN(R^{13a})R^{13b} or —C(NH)NH₂;
R¹¹ represents H, C₁₋₆ alkyl, -E-aryl or —C(O)R^{12d};
R^{12a} to R^{12d} independently represent, at each occurrence when used herein, C₁₋₆ alkyl (optionally substituted by one or more substituents selected from halo, aryl and Het²), aryl, Het³, or R^{12a} and R^{12d} independently represent H;
R^{13a} and R^{13b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;
E represents, at each occurrence when used herein, a direct bond or C₁₋₄ alkylene;
p represents 1 or 2;
A represents a direct bond, -J-, -J-N(R^{14a})—, -J-S(O)₂N(R^{14b})—, -J-N(R^{14c})S(O)₂— or -J-O— (in which latter four groups, -J is attached to the oxabispidine ring nitrogen);
B represents —Z—-{[C(O)]ₐC(H)(R^{15a})}_{b}—, —Z—[C(O)]_{c}N(R^{15b})—, —Z—N(R^{15c})S(O)₂—, —Z—S(O)₂N(R^{15d})—, —Z—S(O)ₙ—, —Z—O— (in which latter six groups, Z is attached to the carbon atom bearing R⁶ and R⁷), —N(R^{15e})—Z—, —N(R^{15f})S(O)₂—Z—, —S(O)₂N(R^{15g})—Z— or —N(R^{15h})C(O)O—Z— (in which latter four groups, Z is attached to the R⁸ group);
J represents C₁₋₆ alkylene optionally interrupted by —S(O)₂N(R^{14d})— or —N(R^{14e})S(O)₂— and/or optionally substituted by one or more substituents selected from —OH, halo and amino;
Z represents a direct bond or C₁₋₄ alkylene, optionally interrupted by —N(R¹⁵ⁱ)S(O)₂— or —S(O)₂N(R^{15J})—;
a, b and c independently represent 0 or 1;
n represents 0, 1 or 2;
R^{14a} to R^{14e} independently represent, at each occurrence when used herein, H or C₁₋₆ alkyl;
R^{15a} represents H or, together with a single ortho-substituent on the R⁸ group (ortho-relative to the position at which the B group is attached), R^{15a} represents C₂₋₄ alkylene optionally interrupted or terminated by O, S, N(H) or N(C₁₋₆ alkyl);
R^{15b} represents H, C₁₋₆ alkyl or, together with a single ortho-substituent on the R⁸ group (ortho-relative to the position at which the B group is attached), R^{15b} represents C₂₋₄ alkylene;
R^{15c} to R^{15j} independently represent, at each occurrence when used herein, H or C₁₋₆ alkyl;
R⁸ represents phenyl or pyridyl, both of which groups are optionally substituted by one or more substituents selected from —OH, cyano, halo, nitro, C₁₋₆ alkyl (optionally terminated by —N(H)C(O)OR^{16a}), C₁₋₆ alkoxy, —N(R^{17a})R^{17b}, —C(O)R^{17c}, —C(O)OR^{17d}, —C(O)N(R^{17e})R^{17f}, —N(R^{17g})C(O)R^{17h}, —N(R¹⁷ⁱ)C(O)N(R^{17j})R^{17k}, —N(R^{17m})S(O)₂R^{16b}, —S(O)₂N(R¹⁷ⁿ)R^{17o}, —S(O)₂R^{16c}, —OS(O)₂R^{16d} and/or aryl;
and an ortho-substituent (ortho-relative to the attachment of B) may
(i) together with R^{15a}, represent C₂₋₄ alkylene optionally interrupted or terminated by O, S, N(H) or N(C₁₋₆ alkyl), or
(ii) together with R^{15b}, represent C₂₋₄ alkylene;
R^{16a} to R^{16d} independently represent C₁₋₆ alkyl;
R^{17a} and R^{17b} independently represent H, C₁₋₆ alkyl or together represent C₃₋₆ alkylene, resulting in a four- to seven-membered nitrogen-containing ring;
R^{17c} to R^{17o} independently represent H or C₁₋₆ alkyl;
Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{18a})R^{18b}, —C(O)R^{18c}, —C(O)OR^{18d}, —C(O)N(R^{18e})R^{18f}, —N(R^{18g})C(O)R^{18h}, —S(O)₂N(R¹⁸ⁱ)(R^{18j}) and/or —N(R^{18k})S(O)₂R^{18l}; and
R^{18a} to R^{18l} independently represent C₁₋₆ alkyl, aryl or R^{18a} to R^{18k} independently represent H;
provided that:
(a) when R⁷ represents H or C₁₋₆ alkyl; and A represents -J-N(R^{14a})— or -J-O—, then:
(i) J does not represent C₁ alkylene or 1,1-C₂₋₆ alkylene; and
(ii) B does not represent —N(R^{15b})—, —N(R^{15c})S(O)₂—, —S(O)ₙ—, —O—, —N(R^{15e})—Z, —N(R^{15f})S(O)₂—Z— or —N(R^{15b})C(O)O—Z—; and
(b) when R² represents —OR⁹ or -E-N(R¹⁰)R¹¹ in which E represents a direct bond, then:
(i) A does not represent a direct bond, -J-N(R^{14a})—, -J-S(O)₂—N(R^{14b})— or -J-O—; and
(ii) B does not represent —N(R^{15b})—, —N(R^{15c})S(O)₂—, —S(O)ₙ—, —O—, —N(R^{15e})—Z, —N(R^{15f})S(O)₂—Z— or —N(R^{15h})C(O)O—Z—; and
(c) when A represents -J-N(R^{14c})S(O)₂—, then J does not represent C₁ alkylene or 1,1-C₂₋₆ alkylene; and
(d) when R³ represents H or C₁₋₆ alkyl and A represents -J-S(O)₂N(R^{14b})—, then B does not represent —N(R^{15b})—, —N(R^{15c})S(O)₂—, —S(O)ₙ—, —O—, —N(R^{15e})—Z—, —N(R^{15f})S(O)₂—Z— or —N(R^{15h})C(O)O—Z—; and
wherein each aryl and aryloxy group, unless otherwise specified, is optionally substituted;
which process comprises:
(I) hydrogenating a sulfonic acid salt of formula II, 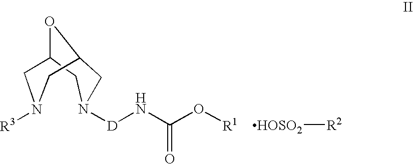
or a solvate thereof;
wherein R¹, R², R³ and D are as defined above, in the presence of a solvent system consisting essentially of water, a C₃₋₅ secondary alkyl alcohol and no more than 15% v/v of another organic solvent; and
(II) without isolating it, reacting the sulfonic acid salt of formula I thereby formed, 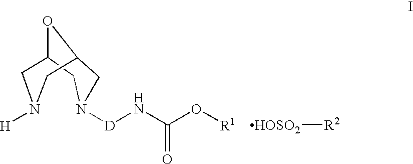
wherein R¹ and D are as defined above with base and
(a) a compound of formula X, 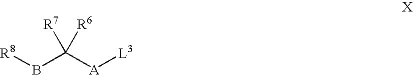
wherein L³ represents a leaving group and R⁶, R⁷, R⁸, A and B are as defined above, or
(b) for compounds of formula IX in which A represents C₂ alkylene and R² and R³ together represent a ═O group, a compound of formula XI, 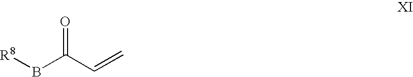
wherein R⁸ and B are as defined above, or
(c) for compounds of formula IX in which A represents CH₂ and R⁶ represents —OH or —N(H)R¹⁰, a compound of formula XII, 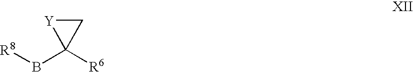
wherein Y represents —O— or —NR¹⁰— and R⁶, R⁸, R¹⁰ and B are as defined above,
wherein the reaction with the compound of formula X, XI or XII is carried out in the presence of a solvent system comprising water and a C₃₋₅ secondary alkyl alcohol.

4. A process as claimed in claim 1, wherein the C₃₋₅ secondary alkyl alcohol is isopropanol.

5. A process as claimed in claim 1, wherein the salt of formula I is produced by catalytically hydrogenating the salt of formula II.

6. A process as claimed in claim 1, wherein hydrogenation of the salt of formula II is performed at 35° C. or above.

7. A process as claimed in claim 1, wherein R¹ represents saturated C₁₋₆ alkyl.

8. A process as claimed in claim 6, wherein R¹ represents tent-butyl.

9. A process as claimed in claim 1, wherein R² represents phenyl, optionally substituted by one or more substituents selected from methyl, halo and nitro.

10. A process as claimed in claim 9, wherein R² represents 2,4,6-trimethylphenyl.

11. A process as claimed in claim 1, wherein R³ represents benzyl, optionally substituted by one or substituents selected from —OH, cyano, halo, nitro, C₁₋₆ alkyl, C₁₋₆ alkoxy, —N(R^{4a})R^{4b}, —C(O)R^{4c}, —C(O)OR^{4d}, —C(O)N(R^{4e})R^{4f}, —N(R^{4g})C(O)R^{4h}, —N(R⁴ⁱ)S(O)₂R^{5a}, —S(O)₂R^{5b} and/or —OS(O)₂R^{5c}, wherein R^{4a} and R^{4b} independently represent H, C₁₋₆ alkyl, or together represent C₃₋₆ alkylene, resulting in a four- to seven-membered nitrogen-containing-ring, R^{4a} to R⁴ⁱ independently represent H or C₁₋₆ alkyl and R^{5a} to R^{5c} independently represent C₁₋₆ alkyl.

12. A process as claimed in claim 11, wherein R³ represents unsubstituted benzyl.

13. A process as claimed in claim 1, wherein the process is performed so as to provide a salt of formula Ia or Ib, 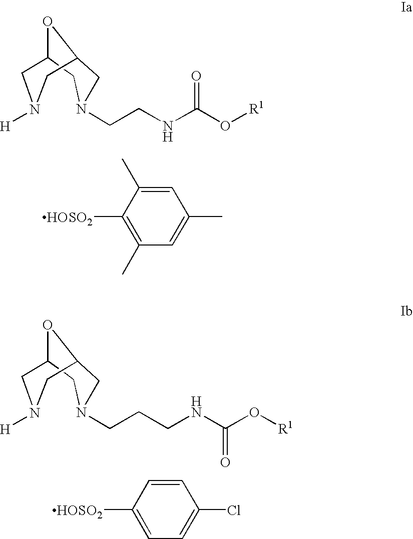
wherein R¹ is as defined in claim 1.

14. A process as claimed in claim 3, wherein step (I) is performed so as to provide a salt of formula Ia or Ib, 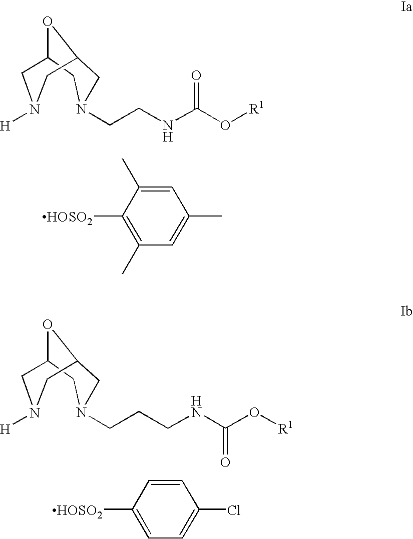
wherein R¹ is represents C₁₋₆ alkyl (optionally substituted by one or more substituents selected from —OH, halo, cyano, nitro and aryl) or aryl.

15. A process as claimed in claim 1, wherein the hydrogenation is performed in the absence of extraneous acids and/or bases.

16. A process as claimed in claim 3, wherein the base employed in step (II) is an alkali metal carbonate.

17. A process as claimed in claim 16, wherein the base is potassium carbonate or sodium carbonate.

18. A process as claimed in claim 3, wherein the reaction of step (II) is between a salt of formula I and a compound of formula XII, as defined in claim 3.

19. A process as claimed in claim 18, wherein the compound of formula XII is 4-(oxiranylmethoxy)benzonitrile.

20. A process as claimed in claim 3, wherein the structural fragment of formula IXa, 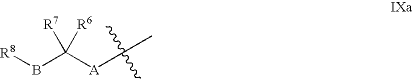
of the compound of formula IX that is ultimately produced represents:

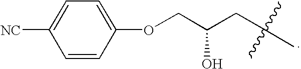

21. A process as claimed in claim 3, wherein the reaction of step (II) is between a salt of formula I and a compound of formula X, as defined in claim 3.

22. A process as claimed in claim 21, wherein the compound of formula X is 4-(2-bromoethoxy)-3-fluorobenzonitrile or 2-(4-cyano-2-fluorophenoxy)ethyl toluene-4-sulfonate.

23. A process as claimed in claim 3, wherein the structural fragment of formula IXa, 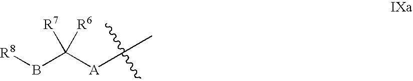
of the compound of formula IX that is ultimately produced represents:

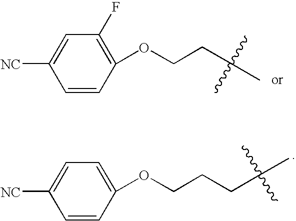

24. A process as claimed in claim 3, wherein, in the compound of formula IX that is produced:
R¹ represents tert-butyl;
D represents —(CH₂)₂— or —(CH₂)₃—;
R⁶ represents H or —OH;
R⁷ represents H;
A represents CH₂;
B represents —Z—O—;
Z represents a direct bond or CH₂; and
R⁸ represents phenyl substituted by cyano in the para-position (relative to B) and optionally substituted by fluoro in the ortho-position (relative to B).

25. A process as claimed in claim 24, wherein the compound of formula IX that is produced is selected from:
tert-butyl 2-{7-[(2S)-3-(4-cyanophenoxy)-2-hydroxypropyl]-9-oxa-3,7-diazabicyclo[3.3.1]non-3-yl}ethylcarbamate;
tert-butyl (2-{7-[2-(4-cyano-2-fluorophenoxy)ethyl]-9-oxa-3,7-diazabicyclo[3.3.1]non-3-yl}ethyl)carbamate; and
tert-butyl (3-{7-[3-(4-cyanophenoxy)propyl]-9-oxa-3,7-diazabicyclo[3.3.1]non-3-yl}propyl)carbamate,
and salts and/or solvates thereof.

26. A mixture consisting essentially of:
(1) an aqueous solution of an alkali metal carbonate; and
(2) 4-(oxiranylmethoxy)benzonitrile.
